(19) United States
(12) Patent Application Publication
(10) Pub. No.: US 2026/0199695 A1
(43) Pub. Date: Jul. 16, 2026

(54) LIGHTWEIGHT NETWORK COMMUNICATIONS FOR WEARABLE CARDIAC DEVICES

(71) Applicant: ZOLL Medical Corporation, Chelmsford, MA (US)

(72) Inventors: Kurt MILLER, Trafford, PA (US); Joshua VAN DER LEE, Mars, PA (US); Shane S. VOLPE, Saltsburg, PA (US); Abhijit SRIBHASHYAM, Pittsburgh, PA (US)

(73) Assignee: ZOLL Medical Corporation, Chelmsford, MA (US)

(21) Appl. No.: 19/139,109

(22) PCT Filed: Dec. 15, 2023
(86) PCT No.: PCT/US2023/084347

§ 371 Date: Jun. 13, 2025

Related U.S. Application Data
(60) Provisional application No. 63/387,785, filed on Dec. 16, 2022.

(51) Int. Cl. A61N 1/39; A61N 1/04; A61N 1/372

(52) U.S. Cl. CPC A61N 1/3904; A61N 1/046; A61N 1/0484; A61N 1/37254; A61N 1/3925

(57) ABSTRACT

A cardiac monitoring system with priority handling of certain clinical and operational communications is provided. The cardiac monitoring system includes an externally worn cardiac device configured to sense one or more electrocardiogram (ECG) signals from a patient wearing the cardiac device. The cardiac device includes a network interface, a memory configured to store BCG data derived from the one or more ECG signals, and a processor coupled with the memory. The processor is configured to determine, from a subset of the ECG data, occurrence of a priority event associated with the cardiac device or the patient wearing the cardiac device, publish, via the network interface, a message specifying the priority event to a first topic using a first communication protocol, and transmit, via the network interface, the ECG data to a remote storage service using a second communication protocol that is different than the first communication protocol.

### CROSS REFERENCE TO RELATED APPLICATION

This application claims the benefit of U.S. Provisional Patent Application 63/387,785 (filed 16 Dec. 2022), the entire disclosure of which is hereby incorporated herein by reference.

### BACKGROUND

The present disclosure is directed to medical devices configured to utilize one or more lightweight communication protocols for patient cardiac telemetry.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation (VF), ventricular tachycardia (VT), pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients who are at risk, have been hospitalized for, or otherwise are suffering from, adverse heart conditions can be prescribed a wearable cardiac monitoring and/or treatment device. In addition to the wearable device, the patient can also be given a battery charger and a set of rechargeable batteries. As the wearable device is generally prescribed for continuous use (e.g., only to be removed when bathing), the patient wears the device during all daily activities such as walking, sitting, climbing stairs, resting or sleeping, and other similar daily activities. Maintaining continuous use of the device as prescribed can be beneficial for monitoring patient progress as well as providing treatment to the patient if needed.

### SUMMARY

In an example, a cardiac system for efficiently publishing ECG data for subscription-based access is provided. The cardiac system includes an externally worn cardiac device configured to sense one or more ECG signals from a skin of a patient. The externally worn cardiac device includes a memory and at least one processor coupled to the memory. The memory is configured to store a device identifier that uniquely identifies the externally worn cardiac device among a plurality of externally worn cardiac devices. The at least one processor configured to subscribe to a device-specific topic that relates to one or more device parameters for controlling operation of the externally worn cardiac device, and publish to a first topic that relates to information derived from the one or more ECG signals sensed by the externally worn cardiac device, the first topic being distinct from the device-specific topic, wherein the device-specific topic incorporates the device identifier.

Examples of the cardiac system may incorporate one or more of the following features.

In the cardiac system, the at least one processor can be further configured to receive, via the device-specific topic, a message specifying one or more device settings associated with the externally worn cardiac device; and apply the one or more device settings to the one or more device parameters of the externally worn cardiac device. The one or more device settings can include a localization setting. The cardiac system can further include a device control service configured to receive input specifying the one or more device settings; generate the message specifying the one or more device settings based on the input; and publish the message to the device-specific topic.

In the cardiac system, the at least one processor can be further configured to subscribe to a patient-specific topic that relates to device parameters for controlling operation of the externally worn cardiac device, the patient-specific topic being distinct from the device-specific topic and the first topic; receive, via the patient-specific topic, a message specifying one or more patient settings associated with the patient; and apply the one or more patient settings to the one or more device parameters of the externally worn cardiac device. The externally worn cardiac device can include one or more monitoring electrodes configured to sense the one or more ECG signals, and one or more therapy electrodes configured to discharge electrotherapy to the patient's skin; and the one or more patient settings can include one or more shock settings assigned to the electrotherapy. The cardiac system can further include a device control service configured to receive input specifying the one or more patient settings; generate the message specifying the one or more patient settings based on the input; and publish the message to the patient-specific topic. In the cardiac system, the at least one processor can be further configured to generate an authentication code; and verify that the message specifying the one or more patient settings can include the authentication code.

In the cardiac system, the externally worn cardiac device can be further configured to sense one or more cardio-acoustic signals from the patient; and the first topic can further relate to information derived from the one or more cardio-acoustic signals sensed by the externally worn cardiac device.

In the cardiac system, the externally worn cardiac device can be further configured to collect device event data indicating a capability of the externally worn cardiac device to monitor and treat the patient; and the first topic further relates to information derived from the device event data collected by the externally worn cardiac device. The device event data can include one or more of a held response button condition, a disconnected therapy electrode condition, or unable to treat condition.

In the cardiac system, the at least one processor can be further configured to derive ECG data from the one or more ECG signals, identify a cardiac arrhythmia condition of the patient indicated within the ECG data, and transmit, using a first communication protocol, the ECG data to a remote storage service; and to publish to the first topic can include to publish, using a second communication protocol distinct from the first communication protocol, a message specifying the cardiac arrhythmia condition of the patient. The device-specific topic can be a first device-specific topic; to transmit the ECG data to the remote storage service can include to publish a message specifying a request for an upload link to a second topic distinct from the first topic and the first device-specific topic, and receive, via a subscription to a second device-specific topic, a message specifying the upload link, the second device-specific topic being distinct from the first topic, the second topic, and the first device-specific topic; and transmit the ECG data to the remote storage service via the upload link.

The cardiac system can further include the remote storage service, wherein the remote storage service can be configured to receive the ECG data using the first communications protocol; and publish a message identifying the ECG data to a second topic using the first communication protocol, the second topic being distinct from the first topic, and the device-specific topic. The message specifying the cardiac arrhythmia condition can further specify an identifier of the ECG data. The cardiac system can further include a message handling service configured to receive the message specifying the cardiac arrhythmia condition of the patient, receive the message identifying the ECG data, and communicate a notification message to a reporting service in response to reception of the message specifying the cardiac arrhythmia condition of the patient.

The cardiac system can further include the reporting service, wherein the report service can be configured to receive the notification message; and communicate an alert message to a recipient process, the alert message specifying a link between the cardiac arrhythmia condition of the patient and the ECG data. In the cardiac system, the externally worn cardiac device can include a security credential created during manufacture of the externally worn cardiac device; and the message handling service can be further configured to register the security credential, and authenticate communications from the externally worn cardiac device using the security credential. The security credential can include the device identifier that uniquely identifies the externally worn cardiac device among a plurality of externally worn cardiac devices; and the message handling service can be further configured to identify the externally worn cardiac device using the device identifier. In the cardiac system, the first communications protocol can be hypertext transfer protocol (HTTP) and the second communications protocol can be message queuing telemetry transport (MQTT).

In another example, a cardiac monitoring system with priority handling of certain clinical and operational communications is provided. The cardiac monitoring system includes an externally worn cardiac device configured to sense one or more electrocardiogram (ECG) signals from a patient wearing the externally worn cardiac device. The externally worn cardiac device includes a network interface, a memory configured to store ECG data derived from the one or more ECG signals, and at least one processor coupled with the memory. The at least one processor is configured to determine, from a subset of the ECG data, occurrence of a priority event associated with the externally worn cardiac device or the patient wearing the externally worn cardiac device, publish, via the network interface, a message specifying the priority event to a first topic using a first communication protocol, and transmit, via the network interface, the ECG data to a remote storage service using a second communication protocol that is different than the first communication protocol.

Examples of the cardiac monitoring system may incorporate one or more of the following features.

In the cardiac monitoring system, the priority event can include one or more of a cardiac arrhythmia condition of the patient and a noise condition of the externally worn cardiac device. The ECG data can include one or more of an ECG segment, a heart rate, a QRS duration, and a QTC interval. The externally worn cardiac device can be further configured to collect device event data indicating a capability of the externally worn cardiac device to discharge electrotherapy to the patient; receive input, via a user interface, indicating that the cardiac arrhythmia condition of the patient is false; and discharge the electrotherapy in response to detection of the cardiac arrhythmia condition and no reception of the input indicating that the cardiac arrhythmia condition of the patient is false. The priority event can be a first priority event; the memory can be further configured to store operational data derived from the device event data, and the at least one processor can be further configured to determine, from a subset of the operational data, occurrence of a second priority event associated with the externally worn cardiac device or the patient wearing the externally worn cardiac device, and publish, via the network interface, a message specifying the second priority event to the first topic using the first communication protocol. The second priority event can include an incapacity condition of the externally worn cardiac device to receive the input indicating that the cardiac arrhythmia condition of the patient is false; or an incapacity condition of the externally worn cardiac device to discharge the electrotherapy in response to detection of the cardiac arrhythmia condition.

In the cardiac monitoring system, the externally worn cardiac device can be further configured to sense one or more cardio-acoustic signals from the patient; the memory can be configured to store cardio-acoustic data derived from the one or more cardio-acoustic signals; to determine occurrence of the priority event can include to determine, from the subset of the ECG data and a subset of the cardio-acoustic data, occurrence of the priority event, and the at least one processor can be further configured to transmit, via the network interface, the cardio-acoustic data to the remote storage service using the second communication protocol. The cardio-acoustic data can include one or more of S1, S2, S3, or S4.

In the cardiac monitoring system, the at least one processor can be further configured to receive, via a subscription to a device-specific topic implemented using the first communication protocol, a message specifying one or more device settings associated with the externally worn cardiac device, the device-specific topic being distinct from the first topic; and apply the one or more device settings to one or more operational parameters of the externally worn cardiac device. The memory can be configured to store a device identifier that uniquely identifies the externally worn cardiac device among a plurality of externally worn cardiac devices; and the at least one processor can be further configured to subscribe to the device-specific topic using the device identifier. The device identifier can be stored in the memory during manufacture of the externally worn cardiac device; and the device-specific topic can include a copy of the device identifier stored in the memory. The at least one processor can be further configured to generate an authentication code; and verify that the message specifying the one or more device settings includes the authentication code. The one or more device settings can include a localization setting.

In the cardiac monitoring system, the at least one processor can be further configured to: receive, via a subscription to a patient-specific topic implemented using the first communication protocol, a message specifying one or more patient settings associated with the patient wearing the externally worn cardiac device, the patient-specific topic being distinct from the first topic; and apply the one or more patient settings to one or more operational parameters of the externally worn cardiac device. The externally worn cardiac device can include one or more monitoring electrodes configured to sense the one or more ECG signals, and one or more therapy electrodes configured to discharge electrotherapy to a skin of the patient, and the one or more patient settings can include one or more shock settings assigned to the electrotherapy.

In the cardiac monitoring system, the message specifying the priority event can further specify an identifier of the ECG data. The cardiac monitoring system can further include the remote storage service, wherein the remote storage service can be configured to receive the ECG data using the second communications protocol; and publish a message identifying the ECG data to a second topic using the first communication protocol, the second topic being distinct from the first topic. The cardiac monitoring system can further include a message handling service configured to receive the message specifying the priority event; receive the message identifying the ECG data; and communicate a notification message to a reporting service in response to reception of the message specifying the priority event. The cardiac monitoring system can further include the reporting service, wherein the reporting service can be configured to communicate, in response to reception of the notification message, an alert message specifying a link between the priority event and the ECG data.

In the cardiac monitoring system, the externally worn cardiac device can include a security credential created during manufacture of the externally worn cardiac device; and the message handling service can be further configured to register the security credential, and authenticate communications from the externally worn cardiac device using the security credential. In the cardiac monitoring system, the security credential can include a device identifier that uniquely identifies the externally worn cardiac device among a plurality of externally worn cardiac devices; and the message handling service can be further configured to identify the externally worn cardiac device using the device identifier.

In the cardiac monitoring system, to transmit the ECG data to the remote storage service can include to publish a message specifying a request for an upload link to a second topic distinct from the first topic, and receive, via a subscription to a device-specific topic implemented using the first communication protocol, a message specifying the upload link, the device-specific topic being distinct from the first topic and the second topic; and transmit the ECG data to the remote storage service via the upload link.

In the cardiac monitoring system, the first communications protocol can be message MQTT, and the second communications protocol can be hypertext transfer protocol (HTTP).

In another example, a cardiac treatment system for use in bandwidth-challenged environments can be provided. The system includes an externally worn cardiac device configured to sense one or more ECG signals from a patient wearing the externally worn cardiac device and discharge electrotherapy in response to detection of an arrhythmia condition occurring in the patient. The externally worn cardiac device includes at least one processor configured to receive, via a subscription to a patient-specific topic implemented in a bandwidth-efficient communication protocol, a first message specifying one or more patient settings associated with the patient, receive, via a subscription to a device-specific topic implemented in the bandwidth-efficient communication protocol, a second message specifying one or more device settings associated with the externally worn cardiac device, apply the one or more of patient settings and the one or more device settings to a plurality of operational parameters of the externally worn cardiac device, and control operation of the externally worn cardiac device based on the plurality of operational parameters.

Examples of the cardiac treatment system may incorporate one or more of the following features.

In the cardiac treatment system, the one or more patient settings can specify one or more of a value of a patient baseline parameter, a value of a lead preference parameter, a value of a ventricular fibrillation rate parameter, a value of a ventricular tachycardia rate parameter, or a value of an electrotherapy energy parameter.

In the cardiac treatment system, the one or more device settings can specify one or more of a value of a localization parameter, a value of message handling service URL, or a value of an account lockout parameter.

The cardiac treatment system can further include a device control service configured to receive input specifying the one or more patient settings and the one or more device settings; generate the first message and the second message based on the input; publish the first message to the patient-specific topic; and publish the second message to the device-specific topic. In the cardiac treatment system, the at least one processor can be further configured to generate a first authentication code and a second authentication code; and verify that the first message includes the first authentication code and that the second message includes the second authentication code.

In the cardiac treatment system, the bandwidth-efficient communication protocol can be MQTT, constrained application protocol (CoAP), advanced message queuing protocol (AMQP), lightweight machine-to-machine protocol (LWM2M), or data distribution service (DDS).

In the cardiac treatment system, to control operation of the externally worn cardiac device can include to derive ECG data from the one or more ECG signals; detect the arrhythmia condition via the ECG data; control discharge of the electrotherapy in response to detection of the arrhythmia condition, control publication of, to a first topic using the bandwidth-efficient communication protocol, a third message specifying the arrhythmia condition of the patient, the first topic being distinct from the patient-specific topic and the device-specific topic; and control transmission of the ECG data to a remote storage service using a transfer protocol that is different from the bandwidth-efficient communication protocol. The transfer protocol can be hypertext transfer protocol (HTTP) or file transfer protocol (FTP). The ECG data can include one or more of an ECG segment, a heart rate, a QRS duration, and a QTC interval. To control operation of the externally worn cardiac device can further include to control acquisition of one or more cardio-acoustic signals from the patient; derive cardio-acoustic data from the cardio-acoustic signals; and control transmission of the cardio-acoustic data to the remote storage service using the transfer protocol. The cardio-acoustic data can include one or more of S1, S2, S3, or S4.

The cardiac treatment system can further include the remote storage service, wherein the remote storage service can be configured to receive the ECG data using the transfer protocol; and publish a fourth message identifying the ECG data to a second topic using the bandwidth-efficient communication protocol, the second topic being distinct from the first topic, the device-specific topic, and the patient-specific topic. The third message specifying the cardiac arrhythmia condition can further specify an identifier of the ECG data. The cardiac treatment system can further include a message handling service configured to receive the third message specifying the cardiac arrhythmia condition of the patient; receive the fourth message identifying the ECG data; and communicate an alert message to a reporting service in response to reception of the third message specifying the cardiac arrhythmia condition of the patient. The cardiac treatment system can further include the reporting service, wherein the reporting service can be configured to communicate, in response to reception of the notification message, an alert message specifying a link between the cardiac arrhythmia condition of the patient and the ECG data. In the cardiac treatment system, the externally worn cardiac device can include a security credential created during manufacture of the externally worn cardiac device; and the message handling service can be further configured to register the security credential, and authenticate communications from the externally worn cardiac device using the security credential.

In the cardiac treatment system, the security credential can include a device identifier that uniquely identifies the externally worn cardiac device among a plurality of externally worn cardiac devices; and the message handling service can be further configured to identify the externally worn cardiac device using the device identifier. In the cardiac treatment system, the device-specific topic is a first device-specific topic; to transmit the ECG data to the remote storage service includes to publish a fourth message specifying a request for an upload link to a second topic distinct from the first topic, the first device-specific topic, and the patient-specific topic, receive, via a subscription to a second device-specific topic, a fifth message specifying the upload link, the second device-specific topic being distinct from the first topic, the second topic, the first device-specific topic, and the patient-specific topic; and transmit the ECG data to the remote storage service via the upload link. In the cardiac treatment system, to control operations of the externally worn cardiac device can further include to collect device event data indicating a capability of the externally worn cardiac device to sense the one or more ECG signals and to discharge the electrotherapy; and publish, to the first topic, a fourth message specifying the device event data collected by the externally worn cardiac device. In the cardiac treatment system, the device event data can include one or more of a held response button condition, a disconnected therapy electrode condition, or unable to treat condition.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 is a schematic diagram of a cardiac monitoring system in accordance with examples disclosed herein.

FIG. 2 is a schematic diagram of some features of the cardiac monitoring system of FIG. 1 in accordance with examples disclosed herein.

FIG. 3 is a schematic diagram of some features of the cardiac monitoring system of FIG. 1 in accordance with examples disclosed herein.

FIG. 4 is a schematic diagram of a controller of a wearable medical device in accordance with examples disclosed herein.

FIGS. 5A and 5B are a sequence diagram illustrating provisioning and configuration processes executed by a cardiac monitoring system in accordance with examples disclosed herein.

FIG. 5C is a flow diagram illustrating a message handling process executed by a cardiac monitoring system in accordance with examples disclosed herein.

FIGS. SD and SE are a sequence diagram illustrating another configuration process executed by a cardiac monitoring system in accordance with examples disclosed herein.

FIG. 5F is a flow diagram illustrating a message handling process executed by a cardiac monitoring system in accordance with examples disclosed herein.

FIGS. 6A and 6C are a sequence diagram illustrating a process of publishing priority events executed by a cardiac monitoring system in accordance with examples disclosed herein.

FIG. 6B is a flow diagram illustrating another message handling process executed by a cardiac monitoring system in accordance with examples disclosed herein.

FIGS. 7A and 7B is a sequence diagram illustrating a process of communicating bulk data executed by a cardiac monitoring system in accordance with examples disclosed herein.

FIG. 7C is a flow diagram illustrating a bulk data file handling process executed by a cardiac monitoring system in accordance with examples disclosed herein.

FIG. 7D is a flow diagram illustrating another message handling process executed by a cardiac monitoring system in accordance with examples disclosed herein.

FIG. 8 is a sequence diagram illustrating a process of controlling remote devices executed by a cardiac monitoring system in accordance with examples disclosed herein.

FIG. 9 is a schematic diagram of a computing device in accordance with examples disclosed herein.

FIGS. 10A-10D illustrate example ambulatory cardiac devices in accordance with examples disclosed herein.

FIG. 11 illustrates an example user interface screen for categorizing events detectable by ambulatory cardiac devices as priority events in accordance with examples disclosed herein.

### DETAILED DESCRIPTION

Due to their mobility, ambulatory medical devices are required to operate within a wide variety of environments that change with regularity. Consider, for example, an ambulatory cardiac device, such as a mobile cardiac telemetry (MCT) device or wearable cardioverter-defibrillator (WCD). Such devices are often prescribed to patients dealing with serious, if not life-threatening, conditions and require continuous use to record accurate electrocardiogram (ECG) information for patient diagnosis and treatment. In the case of a WCD, continuous use protects a patient while the ECG information is being recorded. For these devices, the need for continuous use contributes to diverse operating environments because ambulatory patients wear the devices as they go about their daily activities. Such activities may include sleeping, traveling to work, exercising, and so forth. Each of these activities may take the patient, and thus the medical device, to a new environment with different operating conditions.

The varying operating conditions experienced by ambulatory cardiac devices impose challenges to successful operation. For instance, temperature and humidity can affect the ability of an ambulatory cardiac device to acquire, store, and transmit ECG information regarding the patient, for example, as these conditions can affect the quality of a connection between electrodes of the device and the patient's skin. Similarly, network conditions can affect the quality of a connection between the ambulatory medical device and a network through which data is reported. Example implementations including systems, devices, methods, and computer program products as described herein address these challenges. Ambulatory cardiac devices described herein include features configured to cause the device to efficiently communicate ECG information wirelessly while being worn by the patient in a variety of bandwidth-challenged environments. In this regard, the systems and methods described herein include a message service to monitor and/or manage aspects of a connection between the ambulatory cardiac device and a network access point. The features described herein provide for monitoring and/or managing such aspects so that the ambulatory cardiac device is successfully able to transmit recorded ECG information while navigating bandwidth-challenged environments. Example systems, devices, methods, and computer program products as described herein provide a message service capable of robust operation even where connection strength degrades to a point where the connection becomes bandwidth-challenged (e.g., available bandwidth <0.25 Mbps, <0.5 Mbps, <1 Mbps, <2 Mbps, depending on the amount of data targeted for transfer). In these environments, the message service enables the ambulatory cardiac device to transmit recorded ECG information in a timely manner. Example systems, devices, methods, and computer program products as described herein therefore help reduce potentially harmful impacts to the patient where the ECG information indicates occurrence of a priority event, such as a cardiac arrhythmia condition of the patient or other device critical event, including events that can adversely impact safety crucial functions of the device. In examples, an ambulatory cardiac device as disclosed herein can minimize use of battery power in certain situations, e.g., where the device might need to boost connection strength by increasing power supplied to its radio subsystem, expending power on communications, thereby limiting power available for other medical device functions. As such, implementations as described herein minimize the use of battery power on communications and reduce detrimental impacts particularly where the medical device is a WCD, and thus configured to use battery power to deliver therapeutic pulses (e.g., cardioverting or defibrillating pulses) to the patient if a treatable life-threatening arrhythmia condition (e.g., VT or VF) is detected in the patient. One or more advantages of the improved message service as described herein include the following. The message service as described herein ensures message delivery in various bandwidth challenged environments. For example, ambulatory cardiac devices are moveable objects and moreover are also battery-powered devices. This can result in ambulatory cardiac device connections to network access points becoming unstable in certain environments and for some purposes. In cardiac care settings, where the devices often address life-threatening and safety-critical features, it is desirable to improve reliability of communications. Example message service features as described herein minimize data loss and/or duplication. Another benefit of the message service as disclosed herein is that the service is lightweight. For example, the message service is configured to support increasing number of ambulatory cardiac devices, where each device can be configured for low onboard memory and processing power usage. In this context, the message service described herein is lightweight in that it is well-suited for such ambulatory cardiac devices-much more so than services based on conventional protocols (e.g., the HTTP). This is because, for example, an HTTP header may typically comprise about 8000 bytes, whereas the improved message service as described herein can comprise fewer than about 2 to about 10 bytes. Yet another advantage of the present message service is that it preserves battery power. For example, it is expected that battery power consumption of the present message services when compared to standard HTTP can be on the order of 170-times less energy on 3G networks and 50-times less energy on Wi-Fi networks. As another advantage, the present message service is versatile and can operate on a variety of communication networks, including those based on Internet protocol TCP/IP, or any ordered, lossless, and bi-directional networks. The message service can also operate on non-TCP/IP networks (e.g., ZigBee), UDP, or wireless ad hoc networks, or wireless sensor networks (WSNs).

At least some examples described herein manifest an appreciation for the challenges faced by ambulatory cardiac devices as described above. In these examples, a cardiac monitoring system balances a need to promptly report priority events (e.g., a cardiac arrhythmia condition of the patient or other device critical event, including events that can adversely impact safety crucial functions of the device) with a need to conserve power. Systems, devices, methods, and computer program products described herein provide for an improved message service for facilitating communications between ambulatory cardiac devices and a remote server. For instance, in some examples, a message service as described herein facilitates the reporting of priority events detected by ambulatory cardiac devices via a first pipeline that favors speed of reporting over power efficiency and reports routine events detected by ambulatory cardiac devices via a second pipeline that favors power efficiency over speed of reporting.

To enhance reporting speed and robustness, the first pipeline involves fewer operations and utilizes a bandwidth-efficient protocol, including a publish/subscribe message service such as an MQTT protocol implementation (e.g., based on specifications such as MQTT-SN v1.2, MQTT 3.1, MQTT 3.1.1, or MQTT 5 from the OASIS Message Queuing Telemetry Transport Technical Committee) for communications. Use of a bandwidth-efficient protocol increases the robustness of communication functions with respect to bandwidth-challenged environments. This first pipeline includes a priority pipeline, e.g., for handling priority events such as a cardiac arrhythmia condition of the patient or device critical event, including events that can adversely impact safety crucial functions of the device. The priority events processed via the priority pipeline may include, for example, occurrences of patient conditions (e.g., occurrence of a treatable or non-treatable arrhythmia condition, a syncope episode, etc.) and/or occurrences of device critical conditions (e.g., electrode disconnection from the patient, device critical errors that prevent patient treatment, etc.). Other examples of treatable arrhythmia conditions include bradycardia, tachycardia, and asystole, which can be treated by transcutaneous delivery of pacing pulses to the patient. In implementations, a WCD may treat VF and VT events, and monitor and record ECG information relating to bradycardia, tachycardia, and/or asystole. For example, a WCD that monitors for bradycardia, tachycardia, and/or asystole may provide alerts and notifications concerning these bradycardia, tachycardia, and/or asystole events directly to the patient (e.g., via a user interface module integrated into a WCD monitor, a smart phone, or other electronic device carried by the patient).

In examples, non-treatable arrhythmia conditions may also be monitored, including conditions where a device may not treat, but instead monitor and record ECG information for issuing alerts and notifications, and/or for transmitting to remote locations for additional analysis. Examples of these non-treatable arrhythmia conditions include pulseless electrical activity (PEA), cardiac pauses, atrial fibrillation, ectopic beats, premature ventricular contraction (PVC) counts, bigeminy, trigeminy, among others. Examples of device critical errors that can adversely impact safety crucial functions of the device and prevent patient treatment include lack of appropriately deployed (or deployable) conductive gel, and insufficient remaining battery power, among others.

In some examples, the priority pipeline originates with a medical device. In these examples, the medical device transmits a message specifying the priority event to a message service, e.g., the message including, among other details, information indicating a nature of the priority event. For example, the nature of the priority event can be whether the event is a patient priority event or a device priority or critical event. For example, the patient priority event includes events such as life-threatening cardiac arrhythmias occurring in the patient, including VT and/or VF. For example, systems, devices, methods and/or computer program products provided herein include one or more configurable parameters to permit a healthcare provider (HCP), such as an authorized technician, caregiver, or physician to indicate priority events (e.g., via a user interface). For example, a caregiver may use the one or more configurable parameters to indicate that the patient priority event includes events such as cardiac arrhythmia events of the patient, including bradycardia onset, tachycardia onset, and/or asystole events. For example, the device priority or critical event includes device critical errors as noted above that can affect safety function of the device and/or impact on the ability of the device to provide life-saving treatment to the patient. Examples of such device critical errors include detection of malfunction in the gel deployment system, electrode falloff or poor body contact issues, insufficient battery power to issue an appropriate treatment, among others. Further examples of these critical errors, as well as diagnostic self-tests that can be used to detect them, are described in U.S. Pat. No. 10,272,010, titled “SYSTEMS AND METHODS FOR TESTING A MEDICAL DEVICE”, issued Apr. 30, 2019, included herein as Appendix A. For example, systems, devices, methods and/or computer program products provided herein include one or more configurable parameters to permit an authorized technician, caregiver, or physician to indicate device priority or critical events (e.g., via a user interface). For example, a caregiver may use the one or more configurable parameters to indicate that the device priority or critical event includes events such as a gel deployment system failure event or an electrode fall off event.

If the message service supports a publication-subscription protocol, such as an MQTT implementation, the medical device publishes the message to a data upload topic to which a record processor and a reporting service is subscribed. The record processor, in turn, processes the message to extract priority data therefrom, and stores the priority data within a data store accessible by the reporting service. For example, the priority data can include ECG data, patient annotation information, time stamps, and other such medically relevant information associated with the cardiac arrhythmia condition of the patient. For example, the priority data can include time stamps, device diagnostics, or technical log information relating to the device critical event, including events that can adversely impact safety crucial functions of the device. The reporting service receives the message, retrieves the priority data, and interoperates with a healthcare provider (HCP) interface program (e.g., a browser-based application, a native application, etc.) to alert an HCP of the priority event. In examples, the reporting service includes functionality for determining the nature of the priority data (e.g., whether patient priority data or device priority data). In examples, the reporting service includes functionality for determining to alert an HCP if the priority event includes a patient priority event. In examples, the reporting service includes functionality for determining to alert a technician or other designated service representative if the priority event includes a device priority or critical event. Additional details regarding the devices and processes that implement the priority pipeline are described further below.

To enhance power efficiency, the second pipeline utilizes power-efficient operations to limit use of other power-inefficient operations. For instance, routine data processed via the second pipeline is both batched (e.g., delayed and collected into dense groups within memory) and compressed into a bulk data file prior to transmission over a radio. By batching the routine data, the medical device is required to utilize the radio to transmit routine data less frequently than would be necessary if no batching was employed. This feature saves substantial amounts of battery power. In addition, by compressing the bulk data file prior to transmission, the radio consumes less power during transmission of the compressed bulk data file than the radio would consume during transmission of an uncompressed bulk data file. This second pipeline includes a routine pipeline, e.g., for handling communications of routine events. Examples of routine events include occurrences of normal patient physiological conditions and satisfactory device operating conditions. More specifically, in some examples, routine data included in the bulk data file includes data descriptive of device wear time, patient body position, and patient heart rate trends.

In some examples, the routine pipeline originates with a medical device. The medical device interoperates with a storage service to upload a bulk data file to a file store. The storage service includes a publisher that monitors the file store for new bulk data files. The publisher extracts individual data records from the bulk data file and transmits a message for each to the message service. If the message service supports a publication-subscription protocol, such as a MQTT, the publisher publishes the messages to a bulk data topic to which the record processor is subscribed. The record processor, in turn, processes the message to extract routine data therefrom, and stores the routine data within a data store accessible by the reporting service.

Other features of the cardiac monitoring systems and methods described herein promote power efficiency and/or robust communication in the face of varying operating environments, among other benefits. For instance, in some examples, the message service is configured to insert, within certain types of messages received from medical devices, an identifier of the medical device that transmitted the message. This feature enables the medical devices to transmit less data within these types of messages, and thus conserve power.

In some examples, the cardiac monitoring system provides subscription-based access to processes hosted on medical devices and processes hosted in a data center environment that are a part of the cardiac monitoring system. This subscription-based access enables one message published to a particular topic to reach many subscribers to the topic, which enables efficient distribution of information.

To enable precise communication while using a subscription-based protocol, some examples disclosed herein implement device-specific and patient-specific topics. These topics enable messages to be sent to a particular ambulatory cardiac device using, for example, the MQTT protocol, which is bandwidth-efficient. This level of precision is required for some types of messages (e.g., messages regarding medical device configuration). In some examples, configuration messages are generated and transmitted by a cloud-based control service. The control service enables HCPs to modify operational parameters of ambulatory cardiac devices remotely via the transmitted messages. In some examples, the HCPs can access the cloud-based service via an HCP interface program that interoperates with the control service to generate the configuration messages.

Examples of ambulatory cardiac device configuration are now discussed. Configuration parameters, including operational parameters, of ambulatory cardiac devices that can be remotely altered using the control service include patient operational parameters and device operational parameters. Examples of patient operational parameters include a patient name, a patient ECG baseline, patient prescription parameters, cardiac rehabilitation prescription parameters, whether the patient is required to complete a health survey and the required frequency thereof, a preferred ECG sensor lead, a patient identifier, a patient language, therapeutic pulse energy levels, sleep mode hours, a sleep mode treatment delay, a speaker volume, a time zone, a threshold number of days between uploads that will result in a warning if transgressed, a ventricular fibrillation threshold rate, a ventricular tachycardia threshold rate, a threat delay time, and whether the patient is required to complete a walk test and the required frequency thereof. Examples of device operational parameters include localization parameters (e.g., a list of available languages, a list of supported time zones), a URL for connecting to the message service, a number of days between diagnostic recordings, physiologic signals (e.g., ECG, cardio-acoustic, etc.) to be recorded, and a threshold number of login attempts that, if transgressed, will cause the ambulatory cardiac device to lockout the account for which the login attempts failed.

In some examples, the ambulatory cardiac device is configured to interoperate with a storage service to upload a detailed data file specifying ECG segments as a supplement to priority data specifying a patient arrhythmia. In these examples, the priority data includes a reference to the detailed data file so that the reporting service can locate the detailed data file if requested to do so by an HCP.

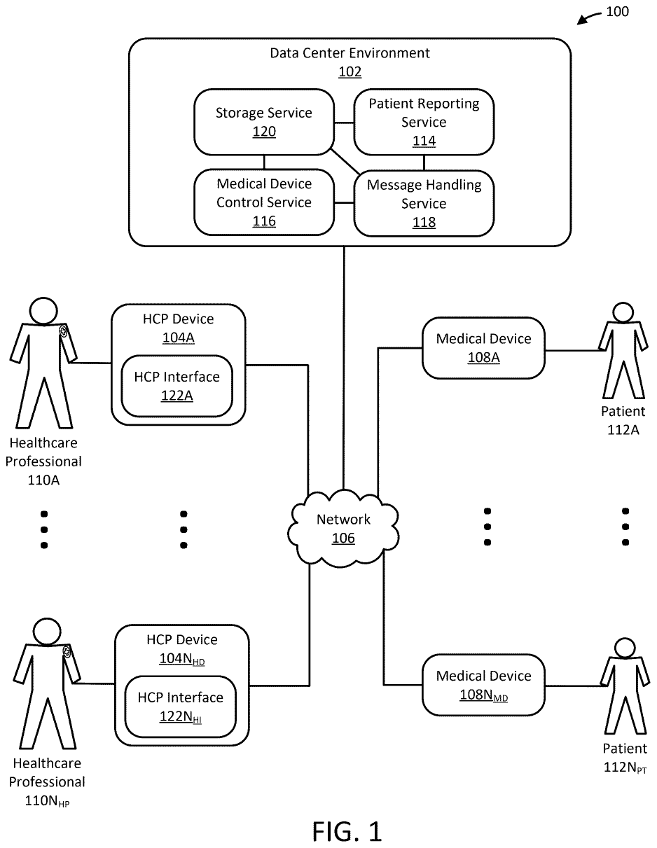

Example systems and methods that implement and provide the foregoing aspects and advantages will now be described in detail with reference to FIGS. 1-11.

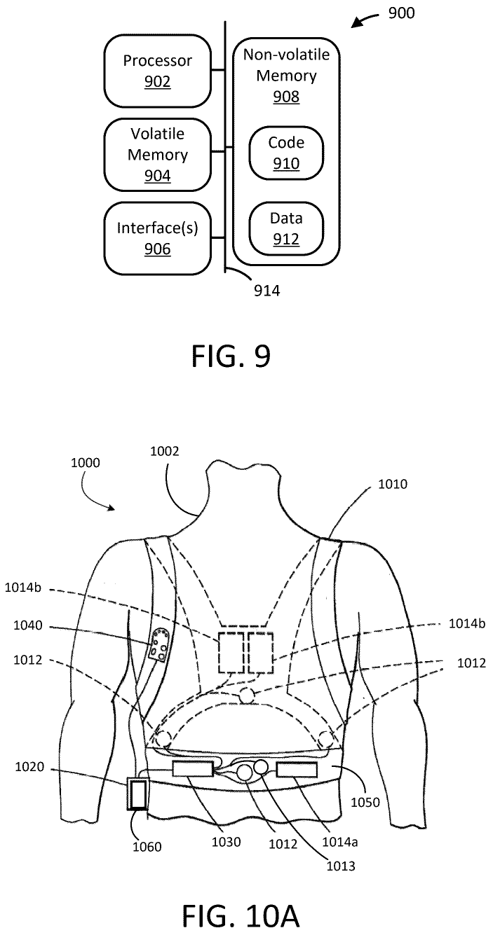

FIG. 1 is a schematic diagram of a cardiac monitoring system 100 configured to monitor and treat patients in accordance with some examples. As shown in FIG. 1, the system 100 includes one or more ambulatory cardiac devices 108A-108N_{MD} (collectively the ambulatory cardiac devices 108), one or more HCP devices 104A-104N_{HD} (collectively the HCP devices 104), a data center environment 102, and a communication network 106. The HCP devices 104 are configured to host one or more HCP interface applications 122A-122N_{HI} (collectively the HCP interface applications 122) and are associated with one or more HCPs 110A-110N_{HP} (collectively the HCPs 110). The ambulatory cardiac devices 108 are associated with, and configured to monitor physiologic data generated by, one or more ambulatory patients 112A-112N_{PT} (collectively the patients 112) as the patients 112 go about their daily activities. As such, in some examples, the ambulatory cardiac devices 108 are wearable by the patients 112. The HCP devices 104, the ambulatory cardiac devices 108, and the data center environment 102 are coupled to, and communicate with one another via, the network 106. Each of the ambulatory cardiac devices 108, the HCP devices 104, the data center environment 102, and the network 106 include one or more computing devices (e.g., as described below with reference to FIG. 9). Associations between the users (e.g., the HCPs 110 and the patients 112) and their devices (e.g., the HCP devices 104 and the ambulatory cardiac devices 108) are established during authentication of the users to the system 100.

As shown in FIG. 1, the data center environment 102 may include physical space, communications, cooling, and power infrastructure to support networked operation of computing devices. For instance, this infrastructure can include rack space into which computing devices are installed, uninterruptible power supplies, cooling plenum and equipment, and networking devices. The data center environment 102 can be dedicated to the cardiac monitoring system 100, can be a non-dedicated, commercially available cloud computing service (e.g., MICROSOFT AZURE, AMAZON WEB SERVICES (AWS), GOOGLE CLOUD, or the like), or can include a hybrid configuration made up of dedicated and non-dedicated resources. Regardless of its physical or logical configuration, as shown in FIG. 1, the data center environment 102 is configured to host a patient reporting service 114, an ambulatory cardiac device control service 116, a message handling service 118, and a storage service 120.

Continuing with the example of FIG. 1, the message service 118 is configured to connect with and route messages between the ambulatory cardiac devices 108, the reporting service 114, the control service 116, and the storage service 120. In operation, the message service 118 implements a secure, scalable, and reliable communication backbone within the system 100.

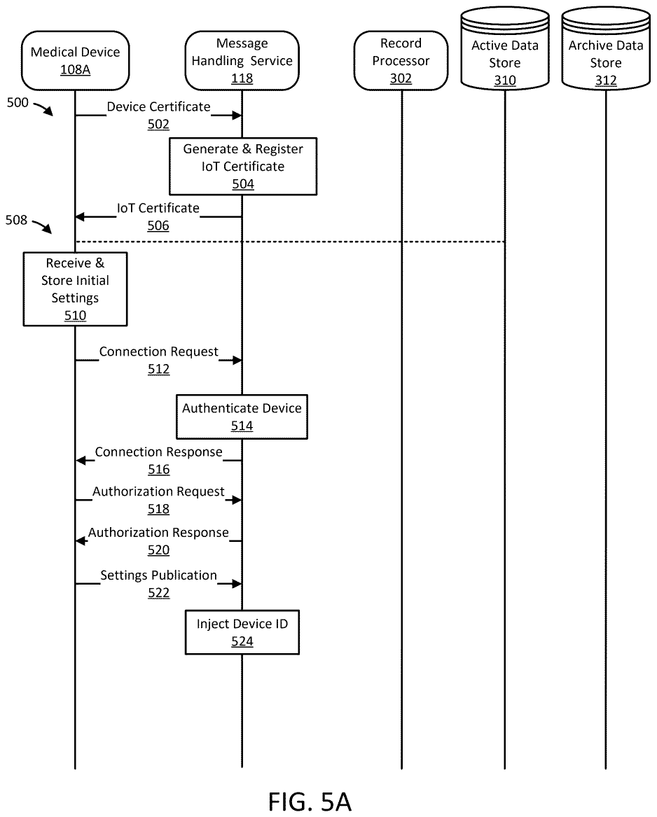

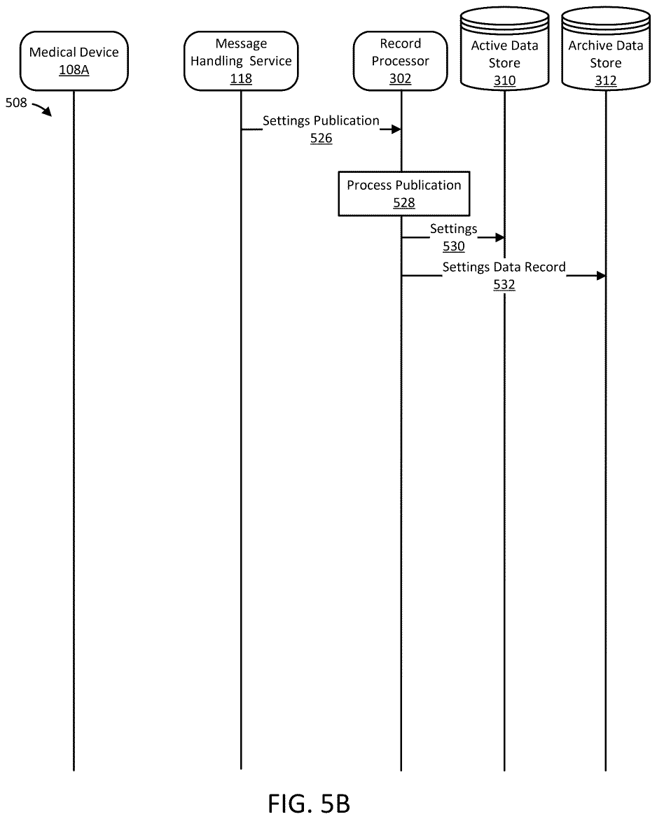

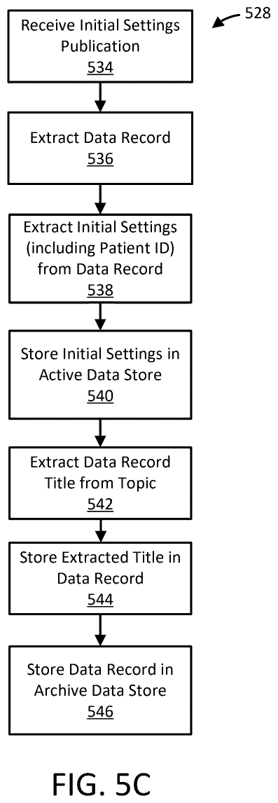

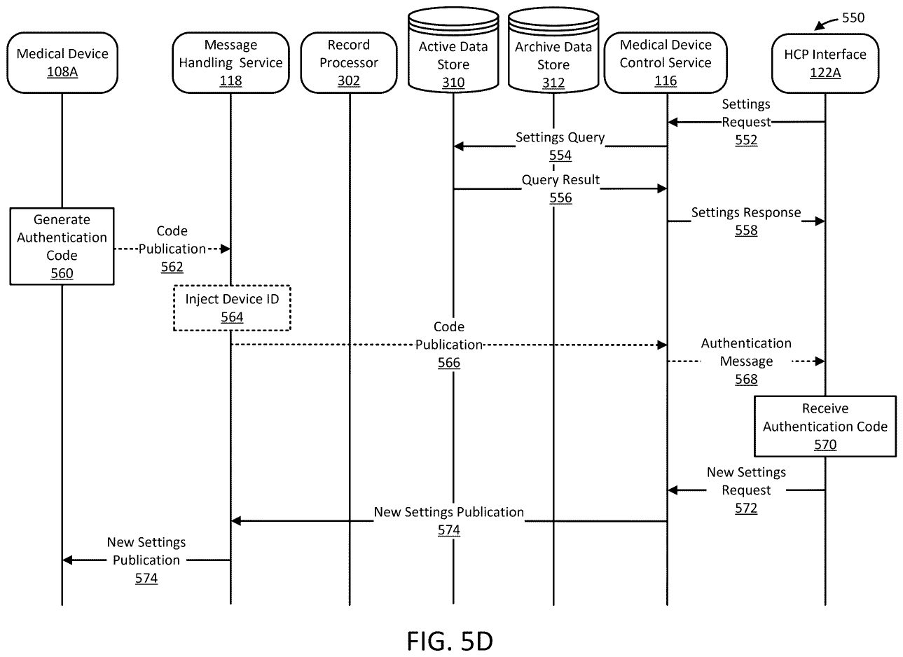

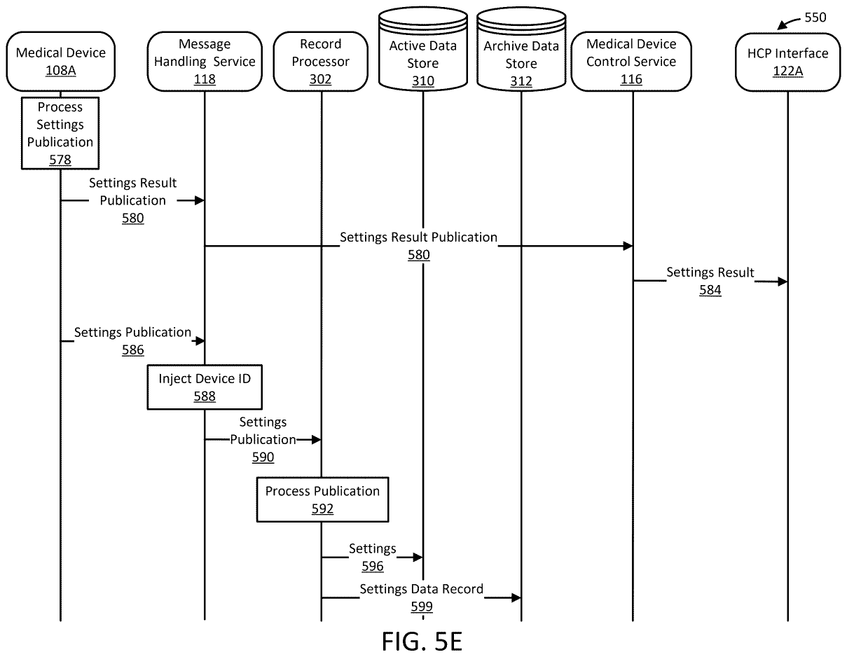

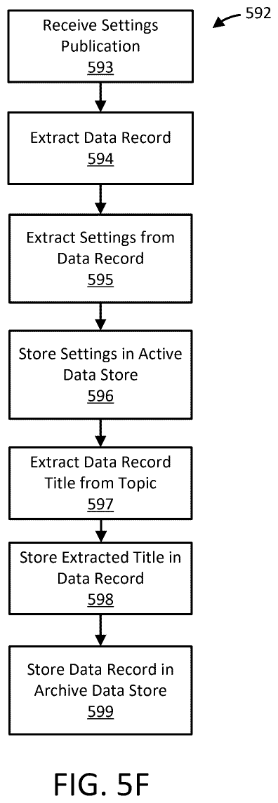

For instance, in some examples, the message service 118 connects to, authenticates, and exchanges messages with the ambulatory cardiac devices 108. The messages exchanged with the ambulatory cardiac devices 108 can specify a broad range of information. For instance, some messages exchanged with the ambulatory cardiac devices 108 include data specifying settings of operational parameters of the ambulatory cardiac devices 108. Other messages include data specifying the operational readiness of the ambulatory cardiac devices 108. Other messages include operational data collected by the ambulatory cardiac devices 108 regarding the ambulatory cardiac devices 108. Other messages can include data specifying requests, generated by the control service 116, for the ambulatory cardiac devices 108 to execute programmatic operations and responses thereto generated by the ambulatory cardiac devices 108. Other messages include data specifying clinical data collected by the ambulatory cardiac devices 108 regarding the patients 112. Other messages can include data requesting one or more links to which the ambulatory cardiac devices 108 may upload one or more files generated by the ambulatory cardiac devices 108. Examples of these and other types of messages are described in further detail below with reference to FIGS. 5A-8.

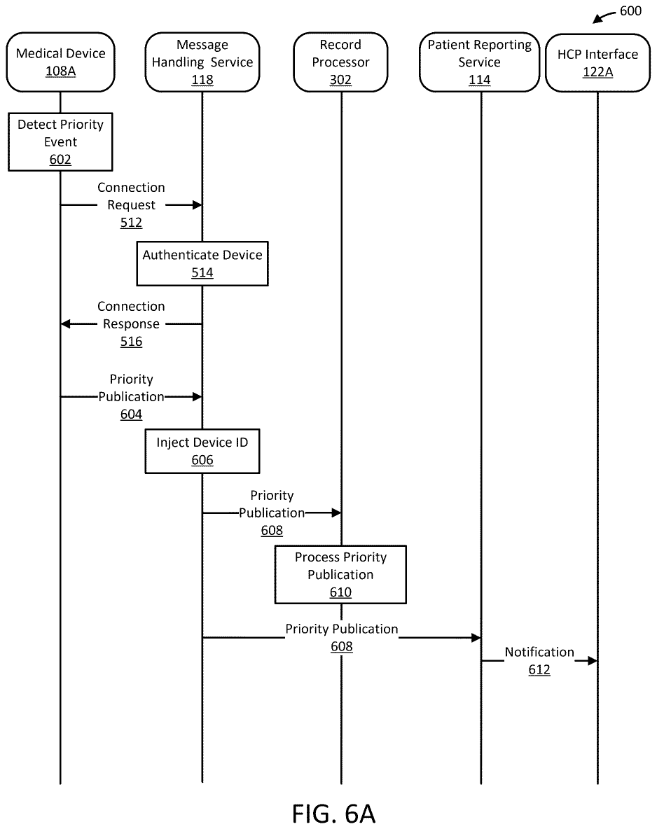

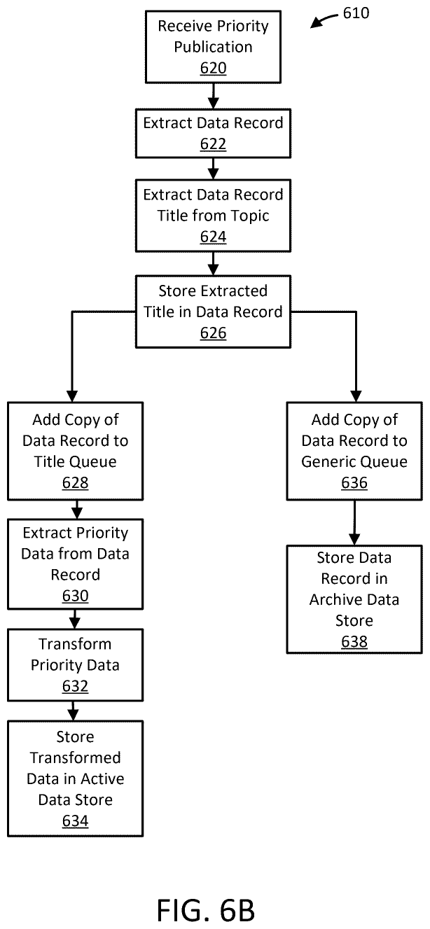

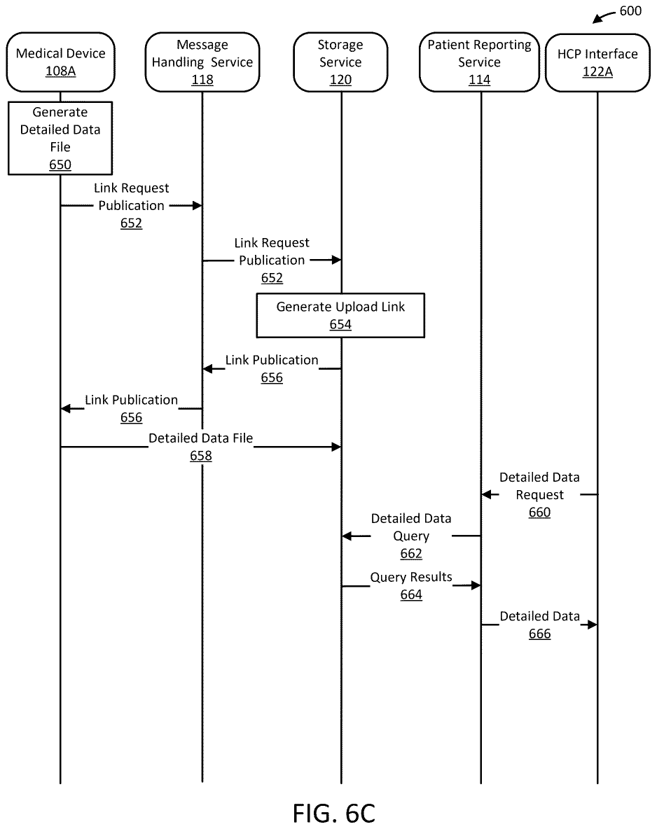

In some examples, the message service 118 exchanges messages with the reporting service 114. These messages can include, for example, data specifying priority events detected by the ambulatory cardiac devices 108. These and other examples of messages exchanged between the message service 118 and the reporting service 114 are described in further detail below with reference to FIGS. 6A and 6C.

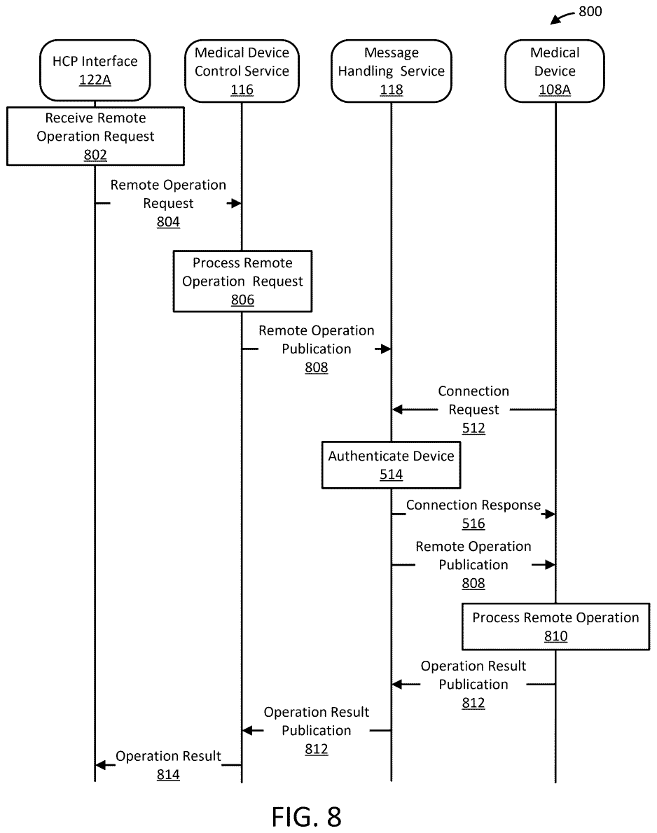

In some examples, the message service 118 exchanges messages with the control service 116. These messages can include, for example, data specifying settings of operational parameters of the ambulatory cardiac devices 108. Other messages can include data specifying requests, generated by the control service 116, for the ambulatory cardiac devices 108 to execute programmatic operations and responses thereto generated by the ambulatory cardiac devices 108. Examples of these and other types of messages are described in further detail below with reference to FIGS. 5D, 5E, and 8.

In some examples, the message service 118 exchanges messages with the storage service 120. These messages can include, for example, data specifying requests for links to storage locations configured to receive and store files generated by the ambulatory cardiac devices 108 and responses to these requests. Other messages can include data specifying settings of operational parameters of the ambulatory cardiac devices 108. Other messages include data specifying the operational readiness of the ambulatory cardiac devices 108. Other messages include operational data collected by the ambulatory cardiac devices 108 regarding the ambulatory cardiac devices 108. Other messages include data specifying clinical data collected by the ambulatory cardiac devices 108 regarding the patients 112. Examples of these and other types of messages are described in further detail below with reference to FIGS. 5A-7D. It should be noted that, in some examples, the messages or data records referred to herein may be written in JavaScript Object Notation (JSON), although other suitable encoding standards will be apparent in view of this disclosure. Because there can be different types of configuration values (e.g., string, integer, float), JSON can be used to store configuration settings, because it is supported by almost every major programming language, and has great support and adoption. In examples, the ambulatory cardiac device can include configuration files that store their current settings in a JSON file. An example JSON record is as below, which presents a periodic heart rate data record.

```
{
  “ts”: 1502473964,
  “uid”: 3,
  “did”: “7138502”, // Added by message
  service based upon the registered
Device ID (not transmitted) (e.g., using identifier injector)
  “wid”: “0247240517138502”,
  “title”: “ECG heart rate data”
  “coeff0”: 1111,
  “coeff1”: 2474,
  “coeff2”: −54875124,
  “coeff3”: 12,
  “coeff4”: 8656,
  “coeff5”: 1,
  “coeff6”: 456
}
```

In some examples, the message service 118 exposes and implements an application programming interface (API) that supports communications via one or more specialized protocols. For instance, in some examples, the message service 118 supports a bandwidth-efficient protocol that requires less traffic and provides greater throughput than hypertext transfer protocol (HTTP). In certain examples, the message service 118 supports a bi-directional protocol that enables duplex communication of packets between devices within a single communication session, unlike HTTP. In certain examples, the message service 118 supports a high-reliability protocol that can guarantee packet delivery subject to time-to-live constraints. In some examples, the message service 118 supports a publish-subscribe protocol that maintains a list of topics to which authenticated processes can publish messages and from which authenticated, subscribed processes can receive messages. In certain examples, the message service 118 exposes and implements an Internet of Things (IoT) protocol that embodies one or more of the specialized protocols enumerated above. Examples of some IoT protocols include MQTT, constrained application protocol (CoAP), advanced message queuing protocol (AMQP), lightweight machine-to-machine protocol (LWM2M), and data distribution service (DDS) to name a few. Support of one or more of the protocols described above enables the ambulatory cardiac devices 108 to communicate effectively with the message service 118 even in bandwidth-challenged environments. Some examples of processes executed by the message service 118 are described further below with reference to FIGS. 5A-8.

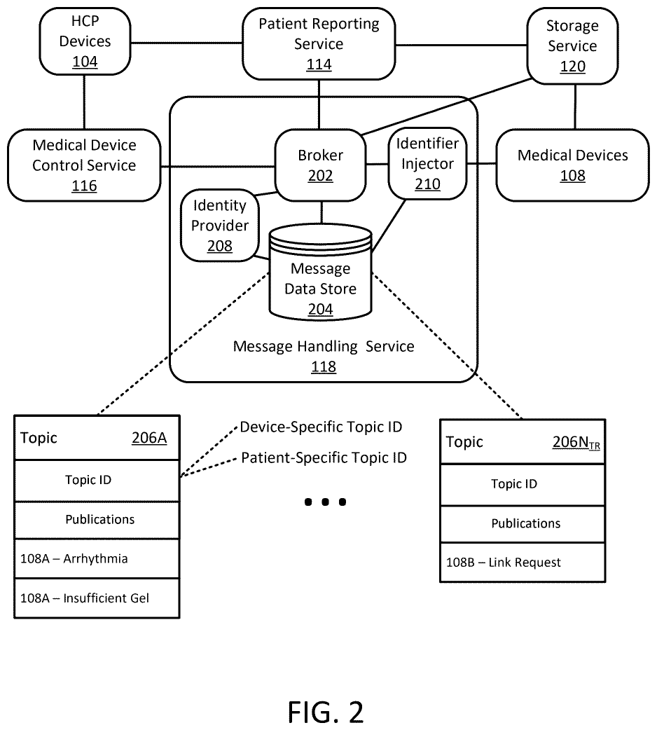

Turning now to FIG. 2, a schematic diagram illustrating additional details regarding an example of the message service 118 is provided. The message service 118 is illustrated in FIG. 2 within the context of the control service 116, the HCP devices 104, the reporting service 114, the storage service 120, and the ambulatory cardiac devices 108 of FIG. 1. The message service 118 includes a broker 202, a message data store 204, an identity provider 208, and an identifier injector 210. In this example, the message service 118 communicates with other processes using a protocol, such as MQTT, that supports subscriptions and publications to topics. As such, the message service 118 is configured to receive messages from one or more publishers (e.g., processes that are authorized within the message service 118 to send messages) that are directed to one or more topics. The message service 118 is further configured to deliver the received messages to subscribers (e.g., processes that are authorized within the message service 118 to subscribe to one or more topics).

As shown in FIG. 2, the message data store 204 stores one or more topic records 206A-206N_{TR} (collectively the topic records 206) that represent the topics supported by the message service 118. Each of the topic records 206 includes a topic ID field and a publications field. A publication includes a message communicated for delivery via a publish-subscribe protocol. The topic ID fields store individual values of topic IDs (e.g., as strings) that uniquely identify each topic. The publications fields store individual copies of, or references to, messages published to the topic identified by the topic ID. The publications fields and a particular topic ID are associated with one another by being stored within the same topic record 206. It should be noted that, in some examples, the publications fields store references (e.g., pointers or some other form of address) to persistent queues, stacks, or other data structures (not shown) that house the publications for the topic identified by the topic ID. In these examples, the message service 118 is configured to allocate and control these queues, stacks, or other data structures. In the example illustrated in FIG. 2, the topic record 206A stores publications directed to a data upload topic for the medical device 108A of FIG. 1, and the topic record 206N_{TR} stores publications directed to link request topic specific to the medical device 108B of FIG. 1. These and other topics are described further below.

In certain examples, at least some topic records 206 within the data store 204 house topic IDs that are specific to individual ambulatory cardiac devices 108. In these examples, each device-specific topic ID uniquely identifies an ambulatory cardiac device among all of the ambulatory cardiac devices 108. Examples of values that may be utilized as device-specific topic IDs include strings that include a serial number of the ambulatory cardiac device and/or strings that include a globally unique identifier (GUID) that is assigned to the ambulatory cardiac device, among other values. Alternatively or additionally, in some examples, at least some topic records 206 within the data store 204 house topic IDs that are specific to individual patients 112. In these examples, each patient-specific topic ID uniquely identifies a patient among all of the patients 112 of FIG. 1. One example of a value that may be utilized as a patient-specific topic ID is a string that includes a government issued identification number of the patient. Another example of such a value is a string that includes a GUID or some randomly generated number that is assigned to the patient that uniquely identifies the patient. Other examples will be apparent in view of this disclosure. It should be noted that randomly generated patient identifiers may offer privacy benefits over government issued identification numbers, depending on the implementation of the system 100.

Continuing with the example of FIG. 2, the identity provider 208 is configured to authenticate ambulatory cardiac devices 108 requesting connections with the message service 118 via security credentials communicated by the ambulatory cardiac devices 108 to the message service 118 in connection requests. For instance, in some examples, the identity provider 208 compares the security credentials to security information stored in the data store 204 and authenticates an ambulatory cardiac device if the security credentials match the security information.

Continuing with the example of FIG. 2, the identifier injector 210 identifies ambulatory cardiac devices 108 connected to the message service 118 via an association between the security credentials of the ambulatory cardiac devices 108 and a device ID stored in the data store 204. In these examples, the identifier injector 210 can manipulate data records stored in messages from the ambulatory cardiac devices 108. This manipulation can include, for example, expanding data stored within the data records and/or supplementing the data stored within the data records with additional data (e.g., adding express copies of metadata, such as a device ID stored in the message data store 204). For instance, in certain examples, the identifier injector 210 adds a device identifier and/or a patient identifier to data records generated by the ambulatory cardiac devices 108. This post-receipt manipulation of the data records by the identifier injector 210 benefits the ambulatory cardiac devices 108 in that the post-receipt manipulation enables the ambulatory cardiac devices 108 to transmit less data (and thus expend less power) per message than would be required if the metadata were transmitted in the data records. This power savings can be especially important where the ambulatory cardiac devices 108 are battery powered.

Continuing with the example of FIG. 2, the broker 202 is configured to connect and exchange messages with the control service 116, the HCP devices 104, the reporting service 114, the storage service 120, and the ambulatory cardiac devices 108. In these examples, a connection with the broker 202 can be established via one or more API calls defined by a protocol implemented by the message service 118. These API calls may be executed by a process requesting a connection with the broker 202 during a handshake procedure executed by the requesting process and the broker 202 to establish connections. In some examples, the broker 202 is configured to interoperate with the identity provider 208 to authenticate a process hosted by one of the ambulatory cardiac devices 108 requesting a connection (e.g., via security credentials passed as part of a connection request) during the handshake process. After a connection is established, the broker 202 may exchange messages with the requesting process using the protocol. In some examples illustrated by FIG. 2, the messages exchanged between the broker and other processes may be publications to topics specified by the topic records 206. As such, the messages can be directed to device-specific and/or patient-specific topics that may include device-specific and/or patient-specific identifiers. In this way, the message service 118 provides a facility through which individual ambulatory cardiac devices 108 and/or individual patients 112 can be targeted as discrete recipients of individual messages even where the protocol implements a publish-subscribe paradigm centered around topics.

In some examples, the broker 202 is configured to receive, process, and respond to authorization requests from the control service 116, the HCP devices 104, the reporting service 114, the storage service 120, and the ambulatory cardiac devices 108. These requests may specify one or more topic IDs, one or more types of communication operations for which authorization specific to the topic ID are requested, and one or more publication quality of service (QoS) levels for which authorization specific to the topic IDs are requested. The types of communication operations for which a process can request to be authorized include publication of messages to a topic, subscription to messages from a topic, or both. The QoS levels for which a process can request authorization include delivery of a message to each subscriber at most once, delivery of a message to each subscriber at least once, and delivery of a message to each subscriber exactly once.

In certain examples, the broker 202 is configured to parse a received authorization request to extract the topic IDs, types of communication operations, and QoS levels sought by the requesting process. In these examples, the broker 202 is also configured to evaluate a preconfigured policy (e.g., one or more predetermined rules) to determine whether the requesting process is authorized to execute the requested types of communication operations at the requested QoS levels for the topic IDs. If the broker 202 determines a requesting process is so authorized, the broker 202 stores a record of the authorization within the data store 204, responds to the authorization request with a positive acknowledgement, and will permit the requesting process to participate in the authorized types of communication operations at the authorized QoS levels for the authorized topic IDs via subsequently received API calls. If the broker 202 determines that the requesting process is not so authorized, the broker 202 responds to the authorization request with an error message indicating lack of authorization. In this way, individual ambulatory cardiac devices 108 can be relegated only to particular topics (e.g., topics specific to the individual ambulatory cardiac device and/or topics specific to a patient associated with the ambulatory cardiac device). This feature enhances data integrity and security by preventing a first ambulatory cardiac device from receiving information regarding a second ambulatory cardiac device and/or preventing a first ambulatory cardiac device from publishing information under the guise of a second ambulatory cardiac device. It should be noted that authorization records may have a limited lifespan and, as such, a requesting process may need to be re-authorized from time to time.

Returning to the example of FIG. 1, the storage service 120 is configured to interoperate with the reporting service 114, the control service 116, the message service 118, and the ambulatory cardiac devices 108 to receive, process, store, and provide access to data records and files generated by the system 100. These data records can include, for example, settings of operational parameters of the ambulatory cardiac devices 108, operational data generated by the ambulatory cardiac devices 108, and clinical data generated by the ambulatory cardiac devices 108. The files that the storage service 120 is configured to store may include detailed operational logs generated by the ambulatory cardiac devices and bulk data files that house multiple individual data records derived from the operational and clinical data generated by the ambulatory cardiac devices 108. In some examples, the storage service 120 is configured to interoperate with the message service 118 to receive messages generated by the ambulatory cardiac devices 108, the reporting service 114, and the control service 116. In some examples, the storage service 120 is further configured to publish messages, receive and respond to queries, and otherwise provide access to the messages and files stored within the storage service 120. Some examples of processes executed by the storage service 120 are described further below with reference to FIGS. 5A-7D.

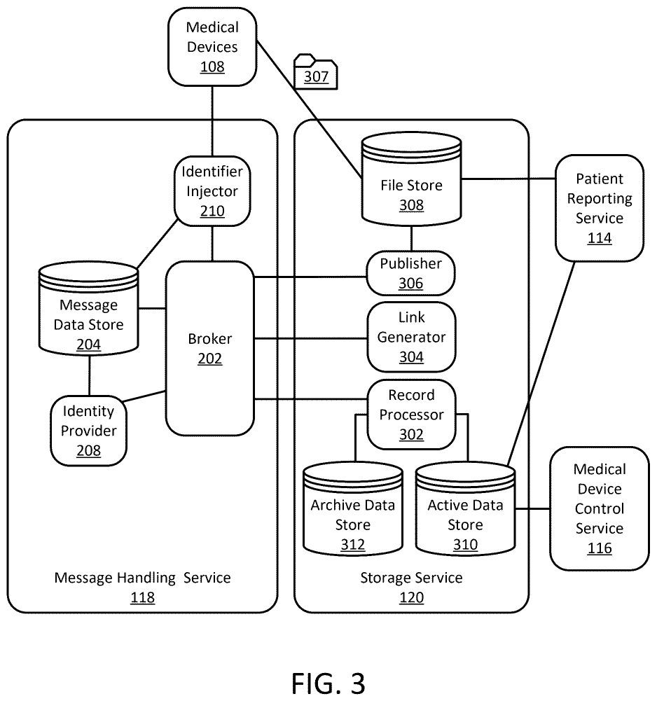

Turning now to FIG. 3, one example of the storage service 120 is illustrated in greater detail. As shown in FIG. 3, the storage service 120 includes a file store 308, a publisher 306, a link generator 304, a record processor 302, an active data store 310, and an archive data store 312. The storage service 120 of FIG. 3 is illustrated within the context of the reporting service 114, the control service 116, the message service 118, and the ambulatory cardiac devices 108.

In at least some examples, the record processor 302 is configured to process messages specifying settings of operational parameters, operational data, and clinical data generated by the ambulatory cardiac devices 108 and received via the broker 202. If the broker 202 supports a publish-subscribe protocol, these messages may be published to one or more topics to which the record processor 302 subscribes. These one or more topics may be directed exclusively to communication of data generated by and/or stored upon the ambulatory cardiac devices 108. In certain examples, the record processor 302 is configured to receive messages, parse the messages to extract one or more data records therefrom, and store copies of the original data records in the archive data store 312 and/or data derived from the data records in the active data store 310 for subsequent interrogation. The derived data stored in the active data store 310 may include operational and clinical data that is accessible to the reporting service 114 and settings of operational parameters that are accessible to the control service 116. In some examples, the record processor 302 generates the derived data by manipulating the data housed in the received data records to ready the data for access by the reporting service 114 and the control service 116. Examples of processes that the record processor 302 is configured to execute are described further below with reference to FIGS. 5A-6B, 7A, 7B, and 7D.

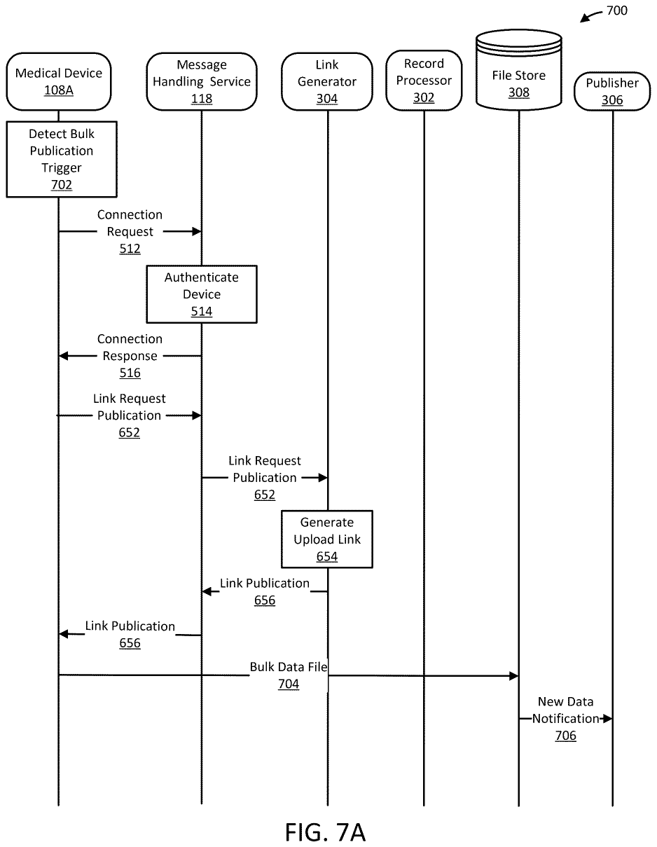

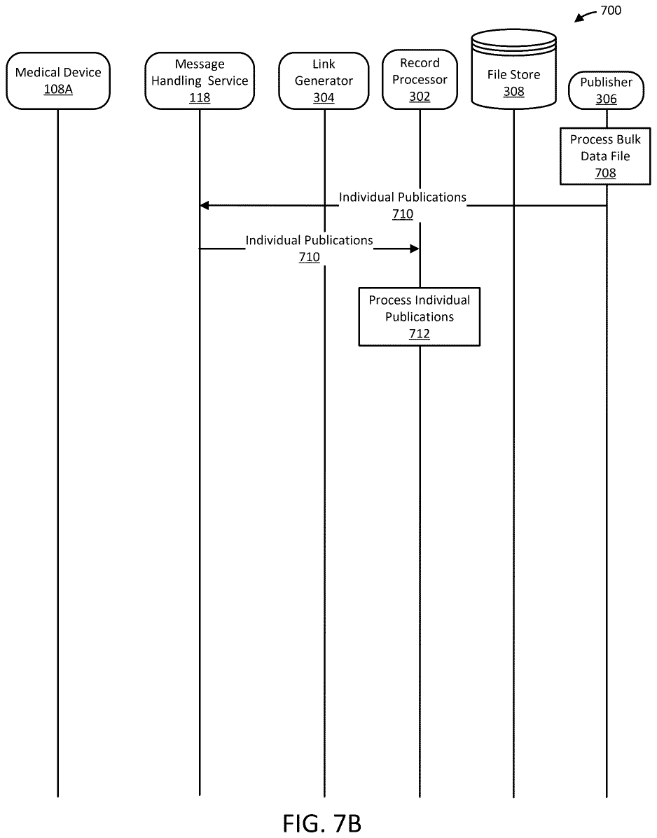

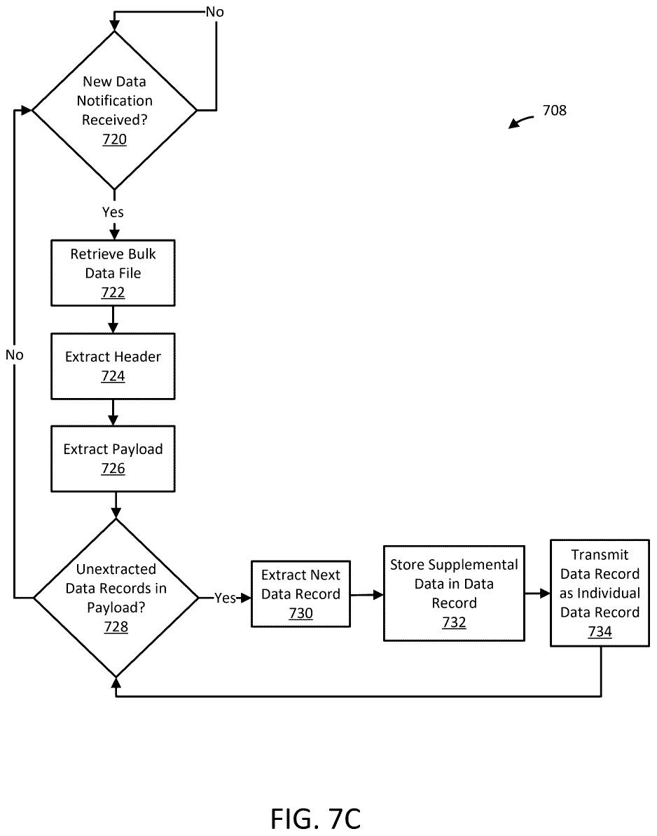

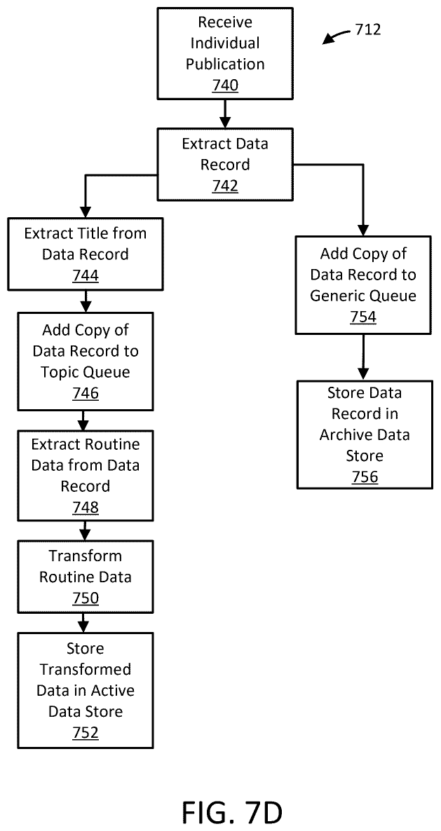

Continuing with the example of FIG. 3, the link generator 304 is configured to process messages specifying upload link requests generated by the ambulatory cardiac devices 108 and received via the broker 202. If the broker 202 supports a publish-subscribe protocol, these messages may be published to one or more topics to which the link generator 304 subscribes. These one or more topics may be directed exclusively to link requests. In certain examples, the link generator 304 is configured to receive a message from a link requestor (e.g., any of the ambulatory cardiac devices 108), parse the message to extract a link request therefrom, generate an upload link, and respond to the link requestor with a response message. This response message may specify the upload link (e.g., uniform resource locator (URL) or other link). The upload link can identify a data store (e.g., the file store 308) that is configured to receive and store a file 307 generated by the link requestor. If the broker 202 supports a publish-subscribe protocol, the link generator 304 may respond to the link requestor by publishing the response message to a topic pertaining to link responses that is specific to the link requestor. This topic may be specified within the link request. Examples of processes that the link generator 304 is configured to execute are described further below with reference to FIGS. 6C and 7A.

Continuing with the example of FIG. 3, the storage service 120 exposes and implements an API configured to receive requests to upload files generated by the ambulatory cardiac devices 108. These files may house, for example, operational and/or clinical data collected by the ambulatory cardiac devices 108. In some examples, to receive the files the storage service 120 implements an HTTP API that utilizes a representational state transfer (REST) architectural style. In these examples, the storage service 120 exposes and monitors one or more HTTP API endpoints as URLs to which the ambulatory cardiac devices 108 can post files for transfer and storage within the storage service 120 (e.g., within the file store 308). In some examples, the URLs made available by the storage service 120 are the URLs generated by the link generator 304 during link request processing. It should be noted that the protocol that underlies the file reception API described above is not limited to HTTP. Other example file reception APIs can utilize other protocols, such as file transfer protocol (FTP) and MQTT among others. It should also be noted that, in at least one example, the file store 308 is implemented as an AMAZON S3 object store. Examples of processes that the storage service 120 is configured to execute that involve the file store 308 are described further below with reference to FIGS. 6C and 7A.

Continuing with the example of FIG. 3, the publisher 306 is configured to process bulk data files that are newly received and stored in the file store 308. In some examples, these bulk data files specify a plurality of individual operational and/or clinical data records generated by the ambulatory cardiac devices 108. The bulk data files may be compressed to reduce resources (e.g., power, bandwidth, etc.) of the ambulatory cardiac devices 108 required to communicate the bulk data files to the file store 308. In some examples, the bulk data file processing executed by the publisher 306 includes monitoring the file store 308 for newly received files that match one or more predetermined properties common to bulk data files (e.g., a filename of a specific type, etc.), decompressing any newly received file that matches the predetermined properties, and parsing the file to extract the individual operational and/or clinical data records housed therein. In certain examples, the processing executed by the publisher 306 further includes generating an individual message for each of the individual data records extracted from the file, and sending the individual messages to the broker 202 for downstream processing (e.g., by the record processor 302). If the broker 202 supports a publish-subscribe protocol, these individual messages may be published to one or more topics to which the record processor 302 subscribes. These one or more topics may be directed exclusively to communication of data generated by and/or stored upon the ambulatory cardiac device 108. Examples of processes that the publisher 306 is configured to execute are described further below with reference to FIGS. 7A-7C.

Returning to the example of FIG. 1, the reporting service 114 is configured to process operational and clinical data received from the ambulatory cardiac devices 108 and to report the operational and clinical data, or data derived therefrom, to the HCPs 110 via the HCP interface applications 122. The operational and clinical data accessed by the reporting service 114 may be stored, for example, in the file store 308 and/or the active data store 310 of FIG. 3. In some examples, the reporting service 114 receives messages from the message service 118 that specify information regarding events detected by the system 100. In these examples, the reporting service 114 interoperates with the storage service 120 to generate and communicate information regarding the detected events to the HCPs 110 via the HCP interface applications 122. Examples of processes that the reporting service 114 is configured to execute are described further below with reference to FIGS. 6A and 6C.

Continuing with the example of FIG. 1, the control service 116 is configured to retrieve settings of configurable, operational parameters of the ambulatory cardiac devices 108 and adjust the parameters to adapt the behavior of the ambulatory cardiac devices 108 to the needs of the patients 112 as determined by the HCPs 110. For instance, in some examples, the control service 116 retrieves the settings from the storage service 120 and publishes messages to the message service 118 that specify adjusted settings. The settings accessed by the control service 116 may be stored in the active data store 310 of FIG. 3. Further, in some examples, the control service 116 receives, from the message service 118, messages published by the ambulatory cardiac devices 108 that specify errors with requested parameter adjustments. Examples of processes that the control service 116 is configured to execute are described further below with reference to FIGS. 5D, 5E, and 8.

Continuing with the example of FIG. 1, the network 106 can include one or more public and/or private networks that support, for example, Internet Protocol (IP). The network 106 may include, for example, one or more local area networks (LANs), one or more personal area networks (PANs), and/or one or more wide area networks (WANs). The LANs can include wired or wireless networks that support various LAN standards, such as a version of IEEE 802.11 or the like. The PANs can include wired or wireless networks that support various PAN standards, such as BLUETOOTH, ZIGBEE, or the like. The WANs can include wired or wireless networks that support various WAN standards, such as the Code Division Multiple Access (CMDA) radio standard, the Global System for Mobiles (GSM) radio standard, or the like. The network 106 connects and enables data communication between the data center environment 102, the HCP devices 104, and the ambulatory cardiac devices 108. In at least some examples, the data center environment 102 includes network equipment (e.g., routers, switches, etc.) that are configured to communicate with the network. 106 and computing devices collocated with or near the network equipment. It should be noted that, in some examples, the network 106 and any network extant within the data center environment 102 support other communication protocols, such as MQTT or other IoT protocols.

Continuing with the example of FIG. 1, the HCP interface applications 122 are configured to control the HCP devices 104 during certain interactions between the HCP devices 104 and the HCPs 110. For instance, in some examples, the HCP interface applications 122 are configured to interoperate with the reporting service 114 and/or the control service 116 and to interact with the HCPs 110 to allow the HCPs 110 to access the reporting and/or control functionality offered by the reporting service 114 and/or the control service 116. The HCP interface applications 122 can be implemented as native applications and/or browser-based applications. Examples of processes that the HCP interface applications 122 are configured to execute are described further below with reference to FIGS. 5D, SE, 6A, 6C, and 8.

It should be noted that, in at least some examples, the services hosted by the data center environment 102 are implemented using AWS IoT service and AMAZON S3 in combination with customized AWS LAMBDA code.

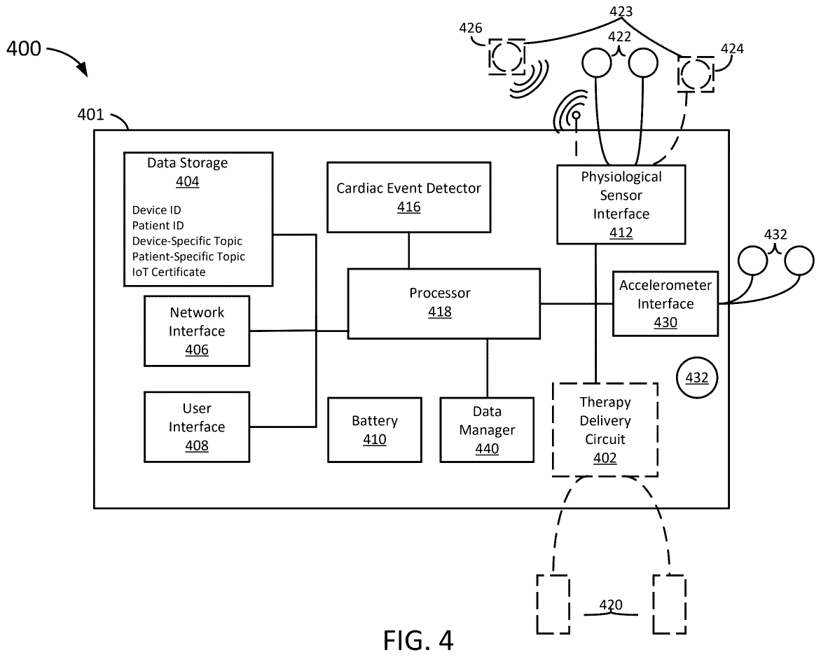

Continuing with the example of FIG. 1, the ambulatory cardiac devices 108 are configured to interoperate with the other parts of the system 100 to monitor and/or treat the patients 112. In certain examples, at least some of the ambulatory cardiac devices 108 are cardiac monitoring and/or treatment devices that incorporate a controller, such as the medical device controller 400 depicted schematically in FIG. 4.

As shown in FIG. 4, the medical device controller 400 can include a housing 401 which can be physically integrated with, or distinct from, other parts of a medical device controlled by the controller 400. The housing 401 can house therapy delivery circuitry 402 configured to provide one or more therapeutic shocks to a patient via at least two therapy electrodes 420, a data storage 404, a network interface 406, a user interface 408, and at least one rechargeable battery 410. The housing 401 can be further configured to house a physiological sensor interface 412, a cardiac event detector 416, a data manager 440, at least one accelerometer 432, an accelerometer interface 430, and at least one processor 418. The physiological sensor interface 412 can be configured to interface with both ECG sensing electrodes 422 and non-ECG physiological sensors 423, such as vibrational sensors, lung fluid sensors, infrared and near-infrared-based pulse oximetry sensors, and blood pressure sensors, among other types of sensors.

In some examples, one or more of the ambulatory cardiac devices 108 includes a medical device controller that includes like components as those described above but that does not include the therapy delivery circuitry 402 and the therapy electrodes 420 (shown in dotted lines). That is, in certain implementations, a medical device can include only ECG monitoring components and not be configured to provide therapy to the patient. In such implementations, such as a heart failure management system (HFMS), a cardiac event monitor (CEM), or a mobile cardiac telemetry (MCT) device, the construction of the controller of the medical device is similar in many respects to the medical device controller 400 but need not include the therapy delivery circuitry 402 and associated therapy electrodes 420.

As further shown in FIG. 4, the therapy delivery circuitry 402 can include, or be operably connected to, circuitry that is configured to generate and provide an electrical therapeutic shock. The circuitry can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuitry and under control of one or more processors (e.g., processor 418) to provide, for example, at least one therapeutic shock to the patient including one or more pacing, cardioversion, or defibrillation therapeutic pulses.

Pacing pulses can be used to treat cardiac arrhythmia conditions such as bradycardia (e.g., less than 30 beats per minute) and tachycardia (e.g., more than 150 beats per minute) using, for example, fixed rate pacing, demand pacing, anti-tachycardia pacing, and the like. Defibrillation pulses can be used to treat ventricular tachycardia and/or ventricular fibrillation.

The capacitors can include a parallel-connected capacitor bank consisting of a plurality of capacitors (e.g., two, three, four or more capacitors). In some examples, the capacitors can include a single film or electrolytic capacitor as a series connected device including a bank of the same capacitors. These capacitors can be switched into a series connection during discharge for a defibrillation pulse. For example, a single capacitor of approximately 140 μF or larger, or four capacitors of approximately 650 μF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 joules of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 joules) regardless of the patient's body impedance. The therapy delivery circuitry 402 can be configured to perform the switching and pulse delivery operations, e.g., under control of the processor 418. As the energy is delivered to the patient, the amount of energy being delivered can be tracked. For example, the amount of energy can be kept to a predetermined constant value even as the pulse waveform is dynamically controlled based on factors such as the patient's body impedance when the pulse is being delivered.

In certain examples, the therapy delivery circuitry 402 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

In some examples, the data manager 440 is configured to store data received, collected, and/or generated by the medical device during operation and to communicate at least some of the stored data to the message service 118 and the storage service 120. Examples of data that the data manager 440 is configured to manipulate include settings of operational parameters, operational data, and clinical data. If received data includes settings of operational parameters, in some examples, the data manager 440 is configured to validate the received settings and apply the settings, if the settings are valid. In applying the settings, the data manager 440 may store values specified by the settings in memory locations referenced by the processor 418 during operation of the medical device to control behavior of the medical device.

In some examples, the data manager 440 is configured to communicate, via the network interface 406, at least some data via one or more compressed files that house a plurality of data records that specify the operational data and clinical data. Alternatively or additionally, in some examples, the data manager 440 is configured to communicate, via the network interface 406, at least some of the data specifying the settings, operational data, and clinical data via one or more messages that house individual data records. These messages can specify a broad range of information. For instance, some messages include data specifying settings of operational parameters of the medical device. Other messages include data specifying the operational readiness of the medical device. Other messages include operational data collected by the medical device regarding the medical device. Other messages include data specifying requests, generated by the control service 116 of FIG. 1, for the medical device to execute programmatic operations and responses thereto generated by the medical device. Other messages include data specifying clinical data collected by the medical device regarding a patient. Other messages can include data requesting one or more links to which the medical device may upload one or more files generated by the medical device.

In some examples, the operational data and/or the clinical data communicated by the data manager 440 is segmented into priority data (e.g., data specifying a priority event) or routine data (e.g., data specifying a routine event). Priority events may include any of an enumerated set of events that are processed by the system 100 using a priority pipeline that favors speed of processing over power efficiency. Examples of priority events include arrhythmia conditions, held response buttons, disconnected electrode conditions, and/or any critical error condition identified by a diagnostic self-test that renders the medical device unable to treat a patient safely. Examples of diagnostic self-tests that the medical device controller is configured to execute to identify and report critical errors as priority events are described further in U.S. Pat. No. 10,272,010. Priority events can be contrasted with routine events, which are processed by the system 100 with a routine pipeline that favors power efficiency of processing over speed. Examples of routine events include normal patient physiological conditions and satisfactory device operating conditions detected with regard to the patients 112 and the ambulatory cardiac devices 108.

In some examples, the data manager 440 is configured to communicate priority data via a priority pipeline and to communicate routine data via a routine pipeline. In these examples, to communicate priority data via the priority pipeline, the data manager 440 packages the priority data into a data record, stores the data record as a payload of a message, and communicates the message to the broker 202 of FIG. 2. The priority data in these messages is processed by the record processor 302 of FIG. 3, stored in the active data store 310 of FIG. 3, and presented to the HCPs 110 of FIG. 1 by the reporting service 114 via the HCP interface applications 122. Further, in these examples, to communicate routine data via the routine pipeline, the data manager 440 packages the routine data into a bulk data file and interoperates with the storage service 120 of FIG. 3 to transmit a compressed version of the bulk data file to the file store 308 of FIG. 3. The routine data in this bulk data file is processed by the publisher 306 of FIG. 3 to generate individual messages that each house an individual data record as a payload. The publisher 306 sends the individual messages to the broker 202 for subsequent processing by the record processor 302, the reporting service 114, and the HCP interface applications 122, as described above with reference to the priority pipeline.

In examples where the broker 202 of FIG. 2 supports a publish-subscribe protocol, the data manager 440 may be configured to communicate messages by publishing the message to one or more topics to which the record processor 302 subscribes. These one or more topics may be directed exclusively to communication of data by the data manager 440. Additionally or alternatively, in these examples, the data manager 440 may be configured to subscribe to one or more device-specific and/or patient-specific topics to ensure messages published to these topics by the control service 116 and the storage service 120 are received by the data manager 440. The topic IDs of the device-specific topics may incorporate an identifier (e.g., a serial number) of the medical device stored in the data storage 404. The topic IDs of the patient-specific topics may incorporate an identifier (e.g., a randomly generated character string) of the patient stored in the data storage 404. Examples of processes that the data manager 440 is configured to execute are described further below with reference to FIGS. 5A-8.

The data manager 440 can be configured to operate under the control of the processor 418 to execute one or more operations as described herein. The data manager 440 can be implemented using hardware or a combination of hardware and software. For instance, in some examples, the data manager 440 can be implemented as code that is stored within the data storage 404 and executed by the processor 418. In this example, the instructions included in the data manager 440 can cause the processor 418 to execute one or more of the operations attributed to the data manager 440 herein. In other examples, the data manager 440 can be an application-specific integrated circuit (ASIC) that is coupled to the processor 418 and configured to execute one or more of the operations attributed to the data manager 440 herein. Thus, examples of the data manager 440 are not limited to a particular hardware or software implementation.

The data storage 404 can include one or more non-transitory computer-readable media, such as flash memory, solid state memory, magnetic memory, optical memory, cache memory, combinations thereof, and others. The data storage 404 can be configured to store code (e.g., executable instructions) and data used for operation of the medical device controller 400. In certain examples, the data storage can include executable instructions that are configured to cause, through their execution, the processor 418 to perform one or more operations. In some examples, the data storage 404 can be configured to store information such as ECG data as received from, for example, the physiological sensor interface 412. Alternatively or additionally, in some examples, the data storage 404 can be configured to store an IoT certificate (e.g., an X.509 certificate) that specifies a device ID (e.g., a serial number of the medical device). In these examples, the message service 118 of FIG. 1 can utilize the IoT certificate to authenticate the medical device and the device identifier embedded within the IoT certificate to uniquely identify the medical device.

In some examples, the network interface 406 can facilitate the communication of information between the medical device controller 400 and one or more other devices or entities over a communications network. For example, where the medical device controller 400 is included in an ambulatory medical device, the network interface 406 can be configured to communicate with a remote computing device such as a remote server or other similar computing device. The network interface 406 can include, for example, communications circuitry for transmitting data in accordance with a BLUETOOTH wireless standard for exchanging such data over short distances to an intermediary device. For example, such an intermediary device can be configured as a base station, a “hotspot” device, a smartphone, a tablet, a portable computing device, and/or other devices in proximity of the wearable medical device including the medical device controller 400. The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a WI-FI communications link based on the IEEE 802.11 standard.

In certain examples, the user interface 408 can include one or more physical interface devices such as input devices, output devices, and combination input/output devices and a software stack configured to drive operation of the devices. These user interface elements can render visual, audio, and/or tactile content. Thus, the user interface 408 can receive input or provide output, thereby enabling a user to interact with the medical device controller 400.

The medical device controller 400 can also include at least one rechargeable battery 410 configured to provide power to one or more integral parts of the medical device controller 400. The rechargeable battery 410 can include a rechargeable multi-cell battery pack. In one example implementation, the rechargeable battery 410 can include three or more 2200 mAh lithium ion cells that provide electrical power to the other parts of the medical device controller 400. For example, the rechargeable battery 410 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 400.

The physiological sensor interface 412 can include physiological signal circuitry that is coupled to one or more sensors configured to monitor one or more physiological parameters of the patient. As shown, the sensors can be coupled to the medical device controller 400 via a wired or wireless connection. The sensors can include one or more ECG sensing electrodes 422, and non-ECG physiological sensors 423 such as vibration sensor 424, tissue fluid monitors 426 (e.g., based on ultra-wide band RF devices), and motion sensors (e.g., accelerometers, gyroscopes, and/or magnetometers). In some implementations, the sensors can include a plurality of conventional ECG sensing electrodes in addition to digital sensing electrodes.

The sensing electrodes 422 can be configured to monitor a patient's ECG information. For example, by design, the digital sensing electrodes 422 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In some examples, the electrodes 422 can be used with an electrolytic gel dispersed between the electrode surface and the patient's skin. In certain implementations, the electrodes 422 can be dry electrodes that do not need an electrolytic material. As an example, such a dry electrode can be based on tantalum metal and having a tantalum pentoxide coating as is described above. Such dry electrodes can be more comfortable for long term monitoring applications.

Referring back to FIG. 4, the vibration sensors 424 can be configured to detect cardiac or pulmonary vibration information. For example, the vibration sensors 424 can detect a patient's heart valve vibration information. For example, the vibration sensors 424 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 424 can also be configured to detect heart wall motion, for instance, by placement of the sensor in the region of the apical beat. The vibration sensors 424 can include a vibrational sensor configured to detect vibrations from a patient's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (e.g., stridor, crackle, etc.). The vibration sensors 424 can also include a multi-channel accelerometer, for example, a three-channel accelerometer configured to sense movement in each of three orthogonal axes such that patient movement/body position can be detected and correlated to detected cardio-vibrational information. The vibration sensors 424 can transmit information descriptive of the cardio-vibrational information to the sensor interface 412 for subsequent analysis.

The tissue fluid monitors 426 can use RF based techniques to assess fluid levels and accumulation in a patient's body tissue. For example, the tissue fluid monitors 426 can be configured to measure fluid content in the lungs, typically for diagnosis and follow-up of pulmonary edema or lung congestion in heart failure patients. The tissue fluid monitors 426 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 426 can transmit information descriptive of the tissue fluid levels to the sensor interface 412 for subsequent analysis.

As further shown in FIG. 4, the controller 400 can further include an accelerometer interface 430 and a set of accelerometers 432. The accelerometer interface 430 can be operably coupled to each of the accelerometers 432 and configured to receive one or more outputs from the accelerometers. The accelerometer interface 430 can be further configured to condition the output signals by, for example, converting analog accelerometer signals to digital signals (if using an analog accelerometer), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In some examples, the accelerometer interface 430 can be configured to filter the signals using a high-pass or band-pass filter to isolate the acceleration of the patient due to movement from the component of the acceleration due to gravity.

Additionally, the accelerometer interface 430 can configure the output for further processing. For example, the accelerometer interface 430 can be configured to arrange the output of an individual accelerometer 432 as a vector expressing the acceleration components of the x-axis, the y-axis, and the z-axis as received from each accelerometer. The accelerometer interface 430 can be operably coupled to the processor 418 and configured to transfer the output signals from the accelerometers 432 to the processor 418 for further processing and analysis.

As described above, one or more of the accelerometers 432 can be integrated into one or more components of a medical device. For example, as shown in FIG. 4, an accelerometer 432 can be integrated into the controller 400. In some examples, an accelerometer 432 can be integrated into one or more of a therapy electrode 420, a sensing electrode 422, a physiological sensor 423, and into other components of a medical device. When controller 400 is included in a hospital wearable defibrillator (HWD), an accelerometer can be integrated into an adhesive ECG sensing and/or therapy electrode patch.

In certain implementations, the cardiac event detector 416 can be configured to monitor a patient's ECG signal for an occurrence of a cardiac event such as an arrhythmia or other similar cardiac event. The cardiac event detector can be configured to operate under control of the processor 418 to execute one or more methods that process received ECG signals from, for example, the sensing electrodes 422 and determine the likelihood that a patient is experiencing a cardiac event. The cardiac event detector 416 can be implemented using hardware or a combination of hardware and software. For instance, in some examples, cardiac event detector 416 can be implemented as code that is stored within the data storage 404 and executed by the processor 418. In this example, the instructions included in the cardiac event detector 416 can cause the processor 418 to perform one or more methods for analyzing a received ECG signal to determine whether an adverse cardiac event is occurring. In other examples, the cardiac event detector 416 can be an application-specific integrated circuit (ASIC) that is coupled to the processor 418 and configured to monitor ECG signals for adverse cardiac event occurrences. Thus, examples of the cardiac event detector 416 are not limited to a particular hardware or software implementation.

In some implementations, the processor 418 includes one or more processors (or one or more processor cores) that each are configured to perform a series of instructions that result in manipulated data and/or control the operation of the other components of the medical device controller 400. In some implementations, when executing a specific process (e.g., cardiac monitoring), the processor 418 can be configured to make specific logic-based determinations based on input data received and be further configured to provide one or more outputs that can be used to control or otherwise inform subsequent processing to be carried out by the processor 418 and/or other processors or circuitry with which processor 418 is communicatively coupled. Thus, the processor 418 reacts to specific input stimulus in a specific way and generates a corresponding output based on that input stimulus. In some example cases, the processor 418 can proceed through a sequence of logical transitions in which various internal register states and/or other bit cell states internal or external to the processor 418 can be set to logic high or logic low. As referred to herein, the processor 418 can be configured to execute a function where software is stored in a data store coupled to the processor 418, the software being configured to cause the processor 418 to proceed through a sequence of various logic decisions that result in the function being executed. The various components that are described herein as being executable by the processor 418 can be implemented in various forms of specialized hardware, software, or a combination thereof. For example, the processor 418 can be a digital signal processor (DSP) such as a 24-bit DSP. The processor 418 can be a multi-core processor, e.g., having two or more processing cores. The processor 418 can be an Advanced RISC Machine (ARM) processor such as a 32-bit ARM processor or a 64-bit ARM processor. The processor 418 can execute an embedded operating system, and include services provided by the operating system that can be used for file system manipulation, display and audio generation, basic networking, firewalling, data encryption and communications.

In some examples, the ambulatory cardiac devices 108 include front-end configurations that use circuitry to accommodate a signal from a high source impedance from the sensing electrode (e.g., having an internal impedance range from approximately 100 kiloohms to one or more megaohms). This high source impedance signal is processed and transmitted to a monitoring device such as processor 418 of the controller 400 as described above for further processing. In certain implementations, the ambulatory cardiac devices 108 comprise a microprocessor or another dedicated processor operably coupled to the sensing electrodes that is configured to receive a common noise signal from each of the sensing electrodes, sum the common noise signals, invert the summed common noise signals and feed the inverted signal back into the patient as a driven ground using, for example, a driven right leg circuit to cancel out common mode signals.

Turning now to FIG. 5A, a provisioning process 500 is illustrated as a sequence diagram. The process 500 can be executed, in some examples, by a medical device (e.g., the medical device 108A of FIG. 1) and a message service (e.g., the message service 118 of FIG. 1). As shown in FIG. SA, the process 500 starts with the medical device transmitting a device certificate 502 to the message service. The certificate 502 may be created by the medical device (e.g., via the openssl utility) and may specify an identifier of the medical device. For instance, in some examples, the certificate 502 is an X.509 certificate with a serial number of the medical device embedded therein as a subject. In certain examples, to transmit the certificate 502 to the message service, the medical device posts, as part of an HTTP request, the certificate 502 to an API endpoint monitored by the message service, although other forms of communication will be apparent in view of this disclosure.

Continuing with the process 500, the message service generates and registers 504 an IoT certificate using the certificate 502. For instance, in some examples, the message service generates a certificate signing request (CSR) that specifies the certificate 502 as a parameter and transmits the CSR to a certificate authority (not shown) and receives an IoT certificate 506 based on the certificate 502 from the certificate authority in response to the CSR. In certain examples, the message service registers the IoT certificate 506 for subsequent use in authenticating and identifying the medical device and transmits the IoT certificate 506 to the medical device. The medical device stores the IoT certificate in local storage for subsequent use in authenticating and identifying the medical device, and the process 500 ends. It should be noted that, in some examples, the process 500 is executed during manufacture of the medical device.

Continuing with FIGS. 5A and 5B, a configuration process 508 is illustrated as a sequence diagram. The process 508 can be executed, in some examples, by a medical device (e.g., the medical device 108A of FIG. 1), a message service (e.g., the message service 118 of FIG. 1), a record processor (e.g., the record processor 302 of FIG. 3), an active data store (e.g., the active data store 310 of FIG. 3), and an archive data store (e.g., the archive data store 312 of FIG. 3). As shown in FIG. 5A, the process 508 starts with the medical device receiving and storing 510 initial settings of operational parameters for the medical device. These initial settings may be prescribed to a patient (e.g., the patient 112A of FIG. 1) and entered by an HCP (e.g., the HCP 110A of FIG. 1) as part of an initial fitting of the medical device to the patient. The initial settings may specify one or more values of patient operational parameters and/or one or more device operational parameters. Examples of patient operational parameters include a patient name, a patient ECG baseline, whether the patient is required to complete a health survey and the required frequency thereof, a preferred ECG sensor lead, a patient identifier, a patient language, therapeutic pulse energy levels, sleep mode hours, a sleep mode treatment delay, a speaker volume, a time zone, a threshold number of days between uploads that will result in a warning if transgressed, a ventricular fibrillation threshold rate, a ventricular tachycardia threshold rate, a threat delay time, and whether the patient is required to complete a walk test and the required frequency thereof. Examples of device operational parameters include a list of available languages, a list of supported time zones, a URL for connecting to a data center environment (e.g., the data center environment 102 of FIG. 1), a number of days between diagnostic recordings, physiologic signals to be recorded (e.g., ECG signals, cardio-vibrational signals, etc.), and a threshold number of login attempts that, if transgressed, will cause the medical device to lock. Example JSON data records for patient and device operational parameters follow.

Continuing with the process 508, the medical device transmits, to the message service, a message 512 that specifies a connection request that complies with an IoT protocol supported by the message service. For instance, in some examples, the message 512 (or another message transmitted as part of a handshake process between the medical device and the message service to establish a connection) includes a copy of an IoT certificate of the medical device. In response to reception of the message 512, the message service attempts to authenticate 514 the medical device. For instance, in one example, the message service authenticates 514 the medical device by validating the IoT certificate and its contents (e.g. a device identifier) vis-à-vis a previously registered IoT certificate accessible to the message service (e.g., via a message data store, such as the data store 204 of FIG. 2). If the message service successfully authenticates 514 the medical device, the message service transmits a positive acknowledgement within a message 516 specifying a connection response to the medical device. If the message service does not successfully authenticate 514 the medical device, the message service transmits an authentication error message within the message 516, and the process 508 ends.

Continuing with the process 508, the medical device transmits a message 518 specifying an authorization request to the message service. In examples where the message service supports an IoT protocol such as MQTT, the medical device transmits the message 518 to a broker (e.g., the broker 202 of FIG. 2). The message 518 can specify one or more topic IDs, one or more types of communication operations for which authorization specific to the topic ID are requested, and one or more publication QoS levels for which authorization specific to the topic IDs are requested. In response, the broker evaluates a preconfigured policy to determine whether the medical device is authorized to execute the requested types of communication operations at the requested QoS levels for the topic IDs and transmits a message 520 specifying an authorization response to the medical device. If the broker determines that the policy evaluates to true for the medical device, the broker stores a record indicating the authorization of the medical device within a data store (e.g., the message data store 204 of FIG. 2) and the message 520 includes a positive acknowledgement. If the broker determines that the policy evaluates to false, the message 520 includes an error message indicating a lack of authorization.

Continuing with the process 508, the medical device transmits, to the message service, a message 522 that specifies the initial settings of the operational parameters of the medical device. In examples where the message service supports an IoT protocol such as MQTT, the medical device publishes the message 522 to the message service under an initial settings topic to which the record processor is subscribed. The payload of the message 522 may include a data record that specifies the initial settings.

Continuing with the process 508, the message service inserts 524 the device ID of the medical device into the message 522. In some examples, the message service (e.g., via the identifier injector 210 of FIG. 2) retrieves the device ID of the medical device from a message data store (e.g., the message data store 204 of FIG. 2). For instance, in certain examples, the message service interrogates the message data store with a query that requests the device ID for the publisher of the message 522 and receives the device ID in response thereto. Next, the message service writes the retrieved device ID into a predefined location within to the header of the data record stored in the message 522, thereby generating a new message 526. This predefined location may be specified by the data type of the data record, which in turn may be identified by the topic ID to which the message 522 was published. The data type of a data record specifies the name, size, type, and location of each field within any data record of the data type.

Continuing with the process 508 with reference to FIG. 5B, the message service transmits the message 526 to the record processor. In examples where the message service supports an IoT protocol such as MQTT, the message service publishes the message 526 to subscribers of the initial settings topic, which include the record processor.

Continuing with the process 508, the record processor processes 528 the message 526. FIG. SC illustrates one example of a message handling process executed by the record processor within the operation 528. As shown in FIG. 5B, the process 528 starts with the record processor receiving 534 the message 526. For instance, in some examples, the record processor may receive, from the message service, the message 526 as a publication to the initial settings topic.

Continuing with the process 528, the record processor parses the message 526 to extract 536 a data record from the message 526. For instance, in some examples, the record processor identifies a data type associated with the initial settings topic and accesses the payload through the data type to extract the data record.

Continuing with the process 528, the record processor extracts 538 the initial settings from the data record. For instance, in some examples, the record processor reads the values of the initial settings from the fields of the extracted data record.

Continuing with the process 528, the record processor stores 540 the extracted initial settings in the active data store. For instance, in some examples, the record processor executes a query that inserts a new record within the active data store that houses the values of the initial settings read from the fields of the extracted data record.

Continuing with the process 528, the record processor extracts 542 a title of the data record from the topic ID of the topic. For instance, in some examples, the record processor parses the topic ID of the initial settings topic and reads the string “initial_settings” from a predefined location within the topic ID.

Continuing with the process 528, the record processor stores 544 the extracted title in a header of the data record. For instance, in some examples, the record processor writes the string “initial_settings” into a predefined location within the header of the data record. This predefined location may be specified by the data type of the data record.

Continuing with the process 528, the record processor stores 546 the data record in the archive data store. For instance, in some examples, the record processor executes a query that inserts a new record within the archive data store that houses the data record including the supplemented data items (e.g., the title and device ID). Subsequent to the operation 546, the process 528 ends.

Returning to the process 508 with reference to FIG. 5B, during execution of the operation 528, the record processor stores 530 the initial settings of operational parameters for the medical device in the active data store and stores 532 a copy of a data record specifying the initial settings of operational parameters for the medical device in the archive data store. Subsequent to the operation 528, the process 508 ends.

In some situations, an HCP (e.g., the HCP 110A of FIG. 1) may wish to change one or more values of a patient operational parameter (i.e., a patient setting) and/or a value of a device operational parameter (i.e., a device setting) of the medical device. When this occurs, the HCP may call a patient (e.g., the patient 112A of FIG. 1) associated with the medical device to gain the patient's cooperation in making the change via a configuration process, such as the configuration process 550 illustrated in FIGS. 5D and 5E as a sequence diagram. The process 550 can be executed, in some examples, by a medical device (e.g., the medical device 108A of FIG. 1), a message service (e.g., the message service 118 of FIG. 1), a record processor (e.g., the record processor 302 of FIG. 3), an active data store (e.g., the active data store 310 of FIG. 3), an archive data store (e.g., the archive data store 312 of FIG. 3), a control service (e.g., the control service 116 of FIG. 1), and an HCP interface (e.g., the HCP interface 122A of FIG. 1). As shown in FIG. 5C, the process 550 starts with the HCP interface transmitting a message 552 specifying a settings request to the control service. For instance, in some examples, the HCP interface transmits the message 552 in response to reception of input from the HCP indicating that the HCP wishes to reconfigure settings of the medical device. The message 552 may be, for example, an API call and may include, for example, an identifier of the medical device (e.g., a serial number) as a parameter.

Continuing with the process 550, the control service 116 processes the settings request. For instance, in some examples, the control service parses the settings request to extract the device ID specified therein, generates a query 554 requesting the settings of the medical device identified by the device ID, and transmits the query 554 to the active data store.

Continuing with the process 550, the active data store processes the query. For instance, in some examples, the active data store receives the query, identifies a row storing the current settings of the medical device identified in the query, generates query results 556 including the identified settings, and transmits the query results 556 to the control service 116.

Continuing with the process 550, the control service 116 generates a message 558 specifying a settings response to the setting request specified in the message 552 and transmits the message 558 to the HCP interface. For instance, in some examples, the control service 116 transmits the message 558 in reply to an API call executed by the HCP interface. The message 558 may include the current settings specified in the query result 556.

Continuing with the process 550, the medical device generates 560 and outputs an authentication code. For instance, in some examples, the HCP instructs the patient to enter input into the medical device that signals the medical device to enter a support mode. In these examples, upon entry into support mode, the medical device generates 560 the authentication code and outputs the authentication code via a user interface (e.g. the user interface 408 of FIG. 4), and the HCP asks the patient to read the authentication code aloud.

Alternatively or additionally, in certain examples, the medical device transmits a message 562 specifying the authentication code to the message service. In examples where the message service supports an IoT protocol such as MQTT, the medical device publishes the message 562 to the message service as a code publication under a support mode topic to which the control service is subscribed. The payload of the message 562 may include a data record that specifies the authentication code. Further, in these examples, the message service inserts 564 the device ID of the medical device into the header of the message 562, thereby generating a modified message 566, and publishes the message 566 to subscribers of the support mode topic, which include the control service. The control service, in turn, extracts the authentication code and the device ID from the message 566, generates an authentication message 568 and transmits the authentication message 568 to the HCP interface via an API call thereto.

Continuing with the process 550, the HCP interface receives 570 the authentication code. For instance, in some examples, the HCP interface receives input specifying the authentication code from the HCP. Alternatively or additionally, in some examples, the HCP interface receives the authentication message 568 from the control service and parses the message 568 to retrieve the authentication code therefrom.

Continuing with the process 550, the HCP interface receives input specifying the new settings from the HCP, generates a message 572 specifying the new settings, and transmits the message 572 to the control service. The new settings may include new patient settings and/or new device settings. In some examples, the HCP interface transmits the message 572 to the control service by executing an API call exposed and implemented by the control service. This API call may include, for example, an identifier of the medical device (e.g., a serial number), the authentication code, and the new settings as parameters.

It should be noted that, in some examples, the authentication code can be a serial number of the medical device, rather than a distinct authentication code. Further, some examples omit all operations and messages between and including the operation 560 and the operation 570. In such examples, the API call executed by the HCP interface to transmit the message 572 omits the authentication code all together.

Continuing with the process 550, the control service processes the message 572 and transmits a message 574 based on the message 572 to the message service. In examples where the message service supports an IoT protocol such as MQTT, the control service generates the modified message 574 and publishes the modified message 574 to the message service under a device-specific remote action topic to which the medical device is subscribed. The payload of the message 574 may include a data record that specifies the authentication code, the new settings, and a response topic to which the control service is subscribed. The topic ID of the response topic may be specific to the medical device.

Continuing with the process 550, the message service receives the message 574 and transmits the message 574 to the medical device. In examples where the message service supports an IoT protocol such as MQTT, the message service publishes the message 574 to the device-specific remote action topic, to which the medical device is subscribed.

Continuing with the process 550 with reference to FIG. 5E, the medical device processes 578 the message 574. For instance, in some examples, the medical device receives the message 574, parses the message to extract the topic ID of the response topic and the settings from a data record housed in the payload of the message 574, validates the settings, and applies the settings, if the settings are valid. In applying the settings, the medical device stores values specified by the settings in memory locations referenced during operation of the medical device to control behavior of the medical device.

Continuing with the process 550, the medical device generates a message 580 specifying a response and transmits the message 580 to the message service. In examples where the message service supports an IoT protocol such as MQTT, the medical device publishes the message 580 to the message service under the response topic. The message 580 may specify results of the operation 578, such as a positive acknowledgement or an error message. Any error message specified by the message 580 may indicate one or more of the settings that caused a failure to apply the settings.

Continuing with the process 550, the message service receives the message 580 and transmits the message 580 to the control service. In examples where the message service supports an IoT protocol such as MQTT, the message service publishes the message 580 to the response topic, to which the control service is subscribed.

Continuing with the process 550, the control service interoperates with the HCP interface to present the results of the new settings request specified in the message 572 to the HCP. For instance, in some examples, the control service generates a message 584 specifying the settings results and transmits the message 584 to the HCP interface via an API call thereto. Further, in some examples, the HCP interface receives the message 584, parses the message 584 to extract the settings results, and renders a human-readable version of the settings results to the HCP.

Continuing with the process 550, the medical device transmits, to the message service, a message 586 that specifies all settings of the operational parameters of the medical device. In examples where the message service supports an IoT protocol such as MQTT, the medical device publishes the message 586 to the message service under a settings topic (e.g., a patient settings topic and/or a device settings topic) to which the message service is subscribed. The payload of the message 586 may include a data record that specifies all of the settings.

Continuing with the process 550, the message service inserts 588 the device ID of the medical device into the message 586 to generate a new message 590. In some examples, the message service (e.g., via the identifier injector 210 of FIG. 2) retrieves the device ID of the medical device from a message data store (e.g., the message data store 204 of FIG. 2). For instance, in certain examples, the message service interrogates the message data store with a query that requests the device ID for the publisher of the message 586 and receives the device ID in response thereto. Next, the message service writes the retrieved device ID into a predefined location within to the header of the data record stored in the message 586. This predefined location may be specified by the data type of the data record, which in turn may be identified by the topic ID to which the message 586 was published.

Continuing with the process 550, the message service transmits the message 590 to the record processor. In examples where the message service supports an IoT protocol such as MQTT, the message service publishes the message 590 to subscribers of the settings topic, which include the record processor.

Continuing with the process 550, the record processor processes 592 the message 590. FIG. 5F illustrates one example of a message handling process executed by the record processor within the operation 592. As shown in FIG. 5F, the process 592 starts with the record processor receiving 593 the message 590. For instance, in some examples, the record processor may receive, from the message service, the message 590 as a publication to the settings topic.

Continuing with the process 592, the record processor parses the message 590 to extract 594 a data record from the message 590. For instance, in some examples, the record processor identifies a data type associated with the settings topic and accesses the payload through the data type to extract the data record.

Continuing with the process 592, the record processor extracts 595 the settings from the data record. For instance, in some examples, the record processor reads the values of the settings from the fields of the extracted data record.

Continuing with the process 592, the record processor stores 596 the extracted settings in the active data store. For instance, in some examples, the record processor executes a query that inserts a new record within the active data store that houses the values of the settings read from the fields of the extracted data record.

Continuing with the process 592, the record processor extracts 597 a title of the data record from the topic ID of the topic. For instance, in some examples, the record processor parses the topic ID of the initial settings topic and reads the string “patient_settings” and/or “device_settings” from a predefined location within the topic ID.

Continuing with the process 592, the record processor stores 598 the extracted title in a header of the data record. For instance, in some examples, the record processor writes the string “patient_settings” and/or “device_settings” into a predefined location within the header of the data record.

Continuing with the process 592, the record processor stores 599 the data record in the archive data store. For instance, in some examples, the record processor executes a query that inserts a new record within the archive data store that houses the data record including the supplemented data items (e.g., the title and device ID). Subsequent to the operation 599, the process 592 ends.

Returning to the process 550 with reference to FIG. 5E, during execution of the operation 592, the record processor stores 596 the settings of operational parameters for the medical device in the active data store and stores 599 a copy of a data record specifying the settings of operational parameters for the medical device in the archive data store. Subsequent to the operation 599, the process 550 ends.

Turning now to FIGS. 6A and 6C a process 600 for reporting a priority event is illustrated as a sequence diagram. The process 600 can be executed, in some examples, by a medical device (e.g., the medical device 108A of FIG. 1), a message service (e.g., the message service 118 of FIG. 1), a record processor (e.g., the record processor 302 of FIG. 3), a reporting service 114, and an HCP interface (e.g., the HCP interface 122A of FIG. 1). As shown in FIG. 6A, the process 600 starts with the medical device detecting 602 a priority event. Examples of priority events that the medical device can detect include an arrhythmia condition in a patient (e.g., the patient 112A of FIG. 1), a held response button condition in the medical device, a disconnected electrode condition in the medical device, or some other medical device condition that prevents the medical device from being able to treat the patient. The detected arrhythmia condition may be treatable (e.g., VT, VF) or not treatable (Asystole). The disconnected electrode condition can be specific to one or more sensing or therapy electrodes.

Continuing with the process 600, the medical device and the message service establish a connection via execution of the operations regarding the messages 512-516 described above with reference to FIG. 5A. If the medical device is not currently authorized to interoperate with the message service, the medical device and the message service further execute the operations regarding the messages 518 and 520 described above with reference to FIG. 5A (not shown in FIG. 6A) to authorize the medical device to interoperate with the message service.

Continuing with the process 600, the medical device transmits, to the message service, a message 604 that specifies an occurrence of a priority event. In examples where the message service supports an IoT protocol such as MQTT, the medical device publishes the message 604 to the message service under a topic specific to the type of priority event detected (e.g., “treatable_arrhythmia”, “nontreatable_arrhythmia”, etc.) to which the record processor is subscribed. The payload of the message 604 may include a data record that specifies the priority event.

Continuing with the process 600, the message service inserts 606 the device ID of the medical device into the message 604. In some examples, the message service (e.g., via the identifier injector 210 of FIG. 2) retrieves the device ID of the medical device from a message data store (e.g., the message data store 204 of FIG. 2). For instance, in certain examples, the message service interrogates the message data store with a query that requests the device ID for the publisher of the message 604 and receives the device ID in response thereto. Next, the message service writes the retrieved device ID into a predefined location within the header of the data record stored in the message 604, thereby generating a new message 608. This predefined location may be specified by the data type of the data record, which in turn may be identified by the topic ID to which the message 604 was published.

Continuing with the process 600, the message service transmits the message 608 to the record processor. In examples where the message service supports an IoT protocol such as MQTT, the message service publishes the message 608 to subscribers of the priority event topic, which include the record processor and the reporting service.

Continuing with the process 600, the record processor processes 610 the message 608. FIG. 6B illustrates one example of a message handling process executed by the record processor within the operation 610. As shown in FIG. 6B, the process 610 starts with the record processor receiving 620 the message 608. For instance, in some examples, the record processor may receive, from the message service, the message 608 as a publication to the priority event topic.

Continuing with the process 610, the record processor parses the message 608 to extract 622 a data record from the message 608. For instance, in some examples, the record processor identifies a data type associated with the priority event topic and accesses the payload through the data type to extract the data record.

Continuing with the process 610, the record processor extracts 624 a title of the data record from the topic ID of the topic. For instance, in some examples, the record processor parses the topic ID of the priority event topic and reads the string “treatable_arrhythmia” from a predefined location within the topic ID.

Continuing with the process 610, the record processor stores 626 the extracted title in a header of the data record. For instance, in some examples, the record processor writes the string “treatable_arrhythmia” into a predefined location within to the header of the data record. This predefined location may be specified by the data type of the data record.

Continuing with the process 610, the record processor adds 628 a copy of the data record to a queue associated with the extracted title and adds 636 a copy of the data record to a generic queue associated with all data records generated by medical devices.

Continuing with the process 610, the record processor extracts 630 the priority data from the data record. For instance, in some examples, the record processor reads the values of the priority data from the fields of the extracted data record. In some examples, the priority data includes summary information regarding the priority event and a link to detailed data regarding the priority event. For instance, in some examples, the summary information may include a type of arrhythmia detected or a description of a device condition that renders the medical device unable to treat the patient. The detailed data may include, for instance, one or more ECG segments or device diagnostic data temporally adjacent to, surrounding, or otherwise relevant to the priority event. The link may include a relative link to the detailed data that can be fully resolved (e.g., by the reporting service) to a location within an active data store (e.g. the data store 310 of FIG. 3). In one example, the relative link includes patient ID, device ID, and a name of a file storing the detailed data.

Continuing with the process 610, the record processor transforms 632 the priority data into a form expected by one or more consumers of the priority data (e.g., the reporting service). This transformation may include changing the type, precision, and relative location of data items stored within the priority data. Completing this transformation readies the priority data for efficient access by the one or more consumers.

Continuing with the process 610, the record processor stores 634 the transformed priority data in the active data store and removes the copy of the data record from the priority event queue. For instance, in some examples, the record processor executes a query that inserts a new record within the active data store that houses the values (and structure, in some examples) of the transformed priority data.

Continuing with the process 610, the record processor stores 638 the extracted data record in an archive data store (e.g., the data store 312 of FIG. 3) and removes the copy of the data record from the generic queue. For instance, in some examples, the record processor executes a query that inserts a new record within the archive data store that houses the data record including the supplemented data items (e.g., the title and device ID). Subsequent to the operation 638, the process 610 ends.

Returning to the process 600 with reference to FIG. 6A, the reporting service generates a message 612 specifying the priority event and transmits the message 612 to the HCP interface. For instance, in some examples, the reporting service transmits the message 612 using a call to an API exposed and implemented by the HCP interface. The message 612 may include a human-readable rendering of the summary information regarding the priority event and a clickable link to the detailed information. The clickable link may be inactive until a message 666 identifying a detailed data file is processed by the storage service. The message 666 and the processing of the detailed data file are described further below with reference to FIG. 6C.

Continuing with the process 600 with reference to FIG. 6C, the medical device generates 650 the detailed data file described above. For instance, in examples where the priority event is an arrhythmia condition, the detailed data file includes an ECG segment or strip that includes ECG data collected within a time window near to and/or including the occurrence of the arrhythmia condition. This time window may have a duration of 30 seconds, 45 seconds, 1 minute, 1.5 minutes, 2 minutes or longer and may be centered over a time at which the arrhythmia condition was declared. In some examples, the time at which the arrhythmia condition was declared may be offset within the time window (e.g., within the first third of a 45 second time window). Other examples will be apparent in view of this disclosure.

Continuing with the process 600, the medical device generates and transmits, to the message service, a message 652 that specifies a request for a file upload link. In examples where the message service supports an IoT protocol such as MQTT, the medical device publishes the message 652 to the message service under a topic specific to upload link requests and to which the storage service is subscribed. The payload of the message 652 may include a response topic to which the medical device is subscribed. The topic ID of the response topic may be specific to the medical device.

Continuing with the process 600, the message service transmits the message 652 to the storage service. In examples where the message service supports an IoT protocol such as MQTT, the message service publishes the message 652 to subscribers of the upload link event topic, which include the storage service.

Continuing with the process 600, the storage service parses the message 652 to extract the link request specified therein and generates 654 (e.g., via execution of the link generator 304 of FIG. 3) an upload link. For instance, in some examples, the storage service allocates storage space within a file store (e.g., the file store 308 of FIG. 3) to receive a file upload, generates a URL specific to the allocated space to serve as the upload link, and monitors for any messages received that are addressed to the URL. In some examples, the storage service also extracts the response topic from the link request.

Continuing with the process 600, the storage service generates and transmits a message 656 to the message service. The message 656 may specify the upload link. In examples where the message service supports an IoT protocol such as MQTT, to transmit the message the storage service publishes, via the message service, the message 656 to subscribers of the response topic, which include the medical device.

Continuing with the process 600, the message service receives and transmits the message 656 to the medical device. In examples where the message service supports an IoT protocol such as MQTT, the message service publishes the message 656 to subscribers of the response topic, which include the medical device.

Continuing with the process 600, the medical device processes the message 656 to transmit the detailed data file 658 to the storage service. For instance, in some examples, the medical device parses the message 656 to extract the upload link specified therein and executes an HTTP post to the upload link that includes the detailed data file 658 as a parameter.

Continuing with the process 600, the HCP interface receives input from an HCP (e.g., the HCP 110A of FIG. 1) that specifies a request for detailed data regarding a priority event. For instance, in at least one example, the HCP interface receives a click on a clickable link as described above with reference to the message 612 with reference to FIG. 6A. In response, the HCP interface generates and transmits a message 660 specifying a request for detailed data regarding the priority event. For instance, in some examples, the HCP interface transmits an API call to the reporting service that includes a patient ID, a device ID, and a name of the detailed data file as parameters.

Continuing with the process 600, the reporting service processes the message 660. For instance, in some examples, the reporting service parses the message 660 to extract the patient ID, the device ID, and the name of the detailed data file specified therein, generates a query 662 requesting the detailed data file based on the extracted parameters, and transmits the query 662 to the storage service.

Continuing with the process 600, the storage service processes the query. For instance, in some examples, the storage service receives and parses the query, interoperates with the file store to identify the detailed data file using the query parameters, generates query results 664 including the detailed data file, and transmits the query results 664 to the reporting service.

Continuing with the process 600, the reporting service processes the query results 664 to generate a message 666 specifying a response to the message 660 and transmits the message 666 to the HCP interface. For instance, in some examples, the reporting service transmits the message 666 in reply to an API call executed by the HCP interface. The response 666 may include the detailed data file specified in the query result 664, or data derived therefrom. Further, in some examples, the HCP interface receives the response 666, parses the response 666 to extract the detailed data file or data derived therefrom, and renders a human-readable version of the extracted data file or derived data to the HCP, and the process 600 ends.

Turning now to FIGS. 7A and 7B a process 700 for bulk data publishing is illustrated as a sequence diagram. The process 700 can be executed, in some examples, by a medical device (e.g., the medical device 108A of FIG. 1), a message service (e.g., the message service 118 of FIG. 1), a link generator (e.g., the link generator 304 of FIG. 3), a record processor (e.g., the record processor 302 of FIG. 3), a file store (e.g., the file store 308 of FIG. 3), and a publisher (e.g., the publisher 306 of FIG. 3). As shown in FIG. 7A, the process 700 starts with the medical device detecting 702 a bulk publication trigger. Examples of bulk publication triggers that the medical device can detect include input received from a user (e.g. the patient 112A of FIG. 1 or the HCP 110A of FIG. 1) specifying a request to execute a data upload and/or occurrence of an event autonomously generated by the medical device, such as expiration of a timer or the size of a bulk data file transgressing a configurable threshold value. The timer may be periodic (e.g., daily) or aperiodic. In some implementations, the medical device can add a random amount of time to the timer to prevent large numbers of medical devices (e.g., the ambulatory cardiac devices 108 of FIG. 1) from attempting to simultaneously publish bulk data via the message service.

Continuing with the process 700, the medical device and the message service establish a connection via execution of the operations regarding messages 512-516 described above with reference to FIG. 5A. If the medical device is not currently authorized to interoperate with the message service, the medical device and the message service further execute the operations regarding messages 518 and 520 described above with reference to FIG. 5A (not shown in FIG. 7A) to authorize the medical device to interoperate with the message service.

Continuing with the process 700, the medical device, the message service, and the link generator (as part of the storage service) create an upload link via execution of the operations regarding messages 652-656 described above with reference to FIG. 6C.

Continuing with the process 700, the medical device processes the message 656 to transmit a bulk data file 704 to the file store, via an API implemented by the storage service. For instance, in some examples, the medical device parses the message 656 to extract the upload link specified therein and executes an HTTP post to the upload link that includes the bulk data file 704 as a parameter. As described above with reference to FIG. 4, the bulk data file 704 can specify routine data collected by the medical device. In some examples, the bulk data file is structured to include a header and a payload. In these examples, the payload includes a plurality of partial data records, and the header includes supplemental data (e.g., device ID, patient ID, etc.) common to all the partial data records that can be used to generate complete data records from the partial data records.

Continuing with the process 700, the file store receives and stores the bulk data file 704 and transmits a message 706 that indicates new data is available for processing by the publisher within the file store. For instance, in some examples, the file store implements a trigger that generates and transmits the message 706. The message 706 may specify an identifier of the new bulk data file that can be used to access a copy thereof via an API exposed and implemented by the file store.

Continuing with the process 700, the publisher monitors for and processes 708 the new bulk data files. FIG. 7C illustrates one example of a bulk data file handling process 708 executed by the publisher within the operation 708. As shown in FIG. 7C, the process 708 starts with the publisher determining 720 whether a new data notification was received from the file store. For instance, in some examples, the publisher determines whether the message 706 has been received. In these examples, if the publisher determines that the message 706 has been received, the publisher proceeds to operation 722. If the publisher determines that the message 706 has not been received, the publisher re-executes the operation 720.

Continuing with the process 708, the publisher parses the message 706 to extract the identifier of the new bulk data file and retrieves 722 the new bulk data file from the file store. For instance, in some examples the publisher generates and transmits a query to the file store with the identifier of the new bulk data file as a parameter. In these examples, the file store processes the query and responds thereto with a copy of the new bulk data file. The publisher receives the copy and proceeds to operation 724.

Continuing with the process 708, the publisher extracts 724 a header from the bulk data file and extracts the supplemental data from the header. For instance, in some examples, the record processor identifies a data type associated with bulk data files and accesses the header through the data type to extract the header and the supplemental data.

Continuing with the process 708, the publisher extracts 726 a payload from the bulk data file. For instance, in some examples, the record processor identifies the data type associated with bulk data files and accesses the payload through the data type to extract the payload.

Continuing with the process 708, the publisher determines 728 whether any partial data records remain unextracted from the payload. For instance, in some examples, the publisher attempts to access (e.g., using the data type of the bulk data file) a next partial data record from the payload. In these examples, if the publisher successfully accesses the next partial data record from the payload, the publisher proceeds to operation 730. If the publisher fails to access the next partial data record (e.g., due to a now empty payload), the publisher returns to the operation 720.

Continuing with the process 708, the publisher extracts 730 the next partial data record from the payload. For instance, in some examples, the publisher uses the data type of the bulk data file to identify and extract the next partial data record from the payload.

Continuing with the process 708, the publisher stores 732 the supplemental data in the next partial data record to generate a complete data record. For instance, in some examples, the publisher uses the data type of the bulk data file to identify and insert the supplemental data into the next partial data record.

Continuing with the process 708, the publisher transmits 734, to a message service (e.g., the message service 118 of FIG. 7B), a message that specifies the complete data record. In examples where the message service supports an IoT protocol such as MQTT, the publisher publishes the message to the message service under a bulk data topic to which a record processor (e.g., the record processor 302 of FIG. 7B) is subscribed. Subsequent to the operation 734, the process 708 returns to the operation 728.

Returning to the process 700, as a result of the operation 708, the publisher transmits multiple messages 710 to the message service. The message service, in turn, transmits the messages 710 to the record processor. In examples where the message service supports an IoT protocol such as MQTT, the message service publishes the messages 710 to subscribers of the bulk data topic, which include the record processor.

Continuing with the process 700, the record processor processes 712 the messages 710. FIG. 7D illustrates one example of a message handling process executed by the record processor within the operation 712. As shown in FIG. 7D, the process 712 starts with the record processor receiving 740 one of the messages 710. For instance, in some examples, the record processor may receive, from the message service, the message 710 as a publication to the bulk data topic.

Continuing with the process 712, the record processor parses the message 710 to extract 742 a data record from the message 710. For instance, in some examples, the record processor identifies a data type associated with the bulk data topic and accesses the payload through the data type to extract the data record.

Continuing with the process 712, the record processor extracts 744 a title of the data record from the data record. For instance, in some examples, the record processor parses the data record and reads the title from a predefined location within the data record, as defined by the data type associated with bulk data records.

Continuing with the process 712, the record processor adds 746 a copy of the data record to a queue associated with the title and adds 754 a copy of the data record to a generic queue associated with all data records generated by medical devices.

Continuing with the process 712, the record processor extracts 748 the routine data from the data record. For instance, in some examples, the record processor reads the values of the routine data from the fields of the extracted data record. In some examples, the routine data extracted from the data record specifies one or more of body positioning information of a patient (e.g., the patient 112A of FIG. 1), heart rate trend information of the patient, or wear time information of the patient.

Continuing with the process 712, the record processor transforms 750 the routine data into a form expected by one or more consumers (i.e., computer-implemented processes) of the routine data (e.g., the reporting service). This transformation may include changing the type, precision, and relative location of data items stored within the priority data. Completing this transformation readies the routine data for efficient access by the one or more consumers.

Continuing with the process 712, the record processor stores 752 the transformed routine data in the active data store and removes the copy of the data record from the priority event queue. For instance, in some examples, the record processor executes a query that inserts a new record within the active data store that houses the values (and structure, in some examples) of the transformed routine data.

Continuing with the process 712, the record processor stores 756 the extracted data record in an archive data store (e.g., the data store 312 of FIG. 3) and removes the copy of the data record from the generic queue. For instance, in some examples, the record processor executes a query that inserts a new record within the archive data store that houses the data record. Subsequent to the operation 756, the process 712 ends.

Returning to the process 700 with reference to FIG. 7B, subsequent to the operation 712, the process 700 ends.

In some situations, an HCP (e.g., the HCP 110A of FIG. 1) may wish to remotely execute an operation on a medical device (e.g., the medical device 108A of FIG. 1). For instance, the HCP may wish to upgrade the software installed on the medical device, clone the medical device, or initiate other remote operations. Turning now to FIG. 8, a remote execution process 800 that can be initiated by the HCP to accomplish this objective is illustrated as a sequence diagram. The process 800 can be executed, in some examples, by the HCP interface, the medical device, a message service (e.g., the message service 118 of FIG. 1), and a control service (e.g., the control service 116 of FIG. 1). As shown in FIG. 8, the process 800 starts with the HCP interface receiving 802 input from an HCP specifying a remote operation request. For instance, in some examples, the input specifies a request for the medical device to execute a software upgrade.

Continuing with the process 800, the HCP interface transmits a message 804 that specifies a remote operation request to the control service. For instance, in some examples, the HCP interface transmits an API call to the control service that includes an identifier of the medical device (e.g., a serial number) and a command string as parameters.

Continuing with the process 800, the control service processes 806 the remote operation request. For instance, in some examples, the control service parses the remote operation request to extract the device ID and command string specified therein. In these examples, the control service generates a message 808 based on the message 804 and transmits the message 808 to the message service. In examples where the message service supports an IoT protocol such as MQTT, the control service generates the message 808 and publishes the message 808 to the message service under a device-specific remote action topic to which the medical device is subscribed. The payload of the message 808 may specify the command string and a response topic to which the control service is subscribed. The topic ID of the response topic may be specific to the medical device.

Continuing with the process 800, the medical device and the message service establish a connection via execution of the operations regarding messages 512-516 described above with reference to FIG. 5A. If the medical device is not currently authorized to interoperate with the message service, the medical device and the message service further execute the operations regarding messages 518 and 520 described above with reference to FIG. 5A (not shown in FIG. 8) to authorize the medical device to interoperate with the message service.

Continuing with the process 800, the message service transmits the message 808 to the medical device. In examples where the message service supports an IoT protocol such as MQTT, the message service publishes the message 808 to subscribers to the device-specific remote action topic, which include the medical device.

Continuing with the process 800, the medical device processes 810 the message 808. For instance, in some examples, the medical device receives the message 808, parses the message to extract the command string housed in the payload of the message 808, validates the command string, and executes the command string, if the command string is valid.

Continuing with the process 800, the medical device generates a message 812 specifying a response and transmits the message 812 to the message service. In examples where the message service supports an IoT protocol such as MQTT, the medical device publishes the message 812 to the message service under the response topic. The message 812 may specify results of the operation 810, such as a positive acknowledgement or an error message.

Continuing with the process 800, the message service receives the message 812 and transmits the message 812 to the control service. In examples where the message service supports an IoT protocol such as MQTT, the message service publishes the message 812 to the response topic, to which the control service is subscribed.

Continuing with the process 800, the control service interoperates with the HCP interface to present the results of the remote operation request received in the operation 802 to the HCP. For instance, in some examples, the control service generates a message 814 specifying the results and transmits the message 814 to the HCP interface via an API call thereto. Further, in some examples, the HCP interface receives the message 814, parses the message 814 to extract the results, and renders a human-readable version of the results to the HCP.

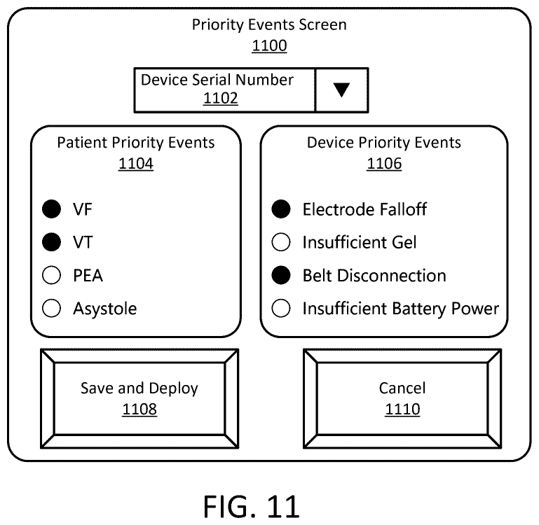

Turning now to FIG. 11, a priority events screen 1100 is illustrated. As shown in FIG. 11, the screen 1100 includes a serial number control 1102, a patient priority events control 1104, a device priority events control 1106, a save control 1108, and a cancel control 1110. In some examples, the priority events screen 1100 is served from a control service (e.g., the control service 116 of FIG. 1) to an HCP interface application (e.g., the HCP interface 122A of FIG. 1). In these examples, the HCP interface application (e.g., a browser) renders the screen 1100. Further, in these examples, the HCP interface application receives input via the screen 1100 from interaction between an HCP (e.g., the HCP 110A of FIG. 1) and the screen 1100.

As shown in FIG. 11, the control 1102 is configured to render one or more serial numbers of one or more ambulatory cardiac devices (e.g., the ambulatory cardiac devices 108 of FIG. 1) under control of the control system. In these examples, responsive to the control 1102 receiving input specifying a serial number of an ambulatory cardiac device for which the HCP wishes to review currently configured priority events, the HCP interface application requests configuration information specifying the configured priority events for the ambulatory cardiac device having the serial number selected in the control 1102. For instance, in some examples, the HCP interface application transmits a settings request (e.g., the settings request 552 of FIG. 5D) to the control service. The control service, in turn, queries an active data store (e.g., the active data store 310) to receive configuration data specifying the currently configured priority events and communicates the configured priority events to the HCP interface application via a settings response (e.g., the settings response 558 of FIG. 5D).

Continuing with the example of FIG. 11, the HCP interface application is configured to render the currently configured priority events via the controls 1104 and 1106. As shown in FIG. 11, the control 1104 indicates that VF and VT are currently configured patient priority events, and the control 1106 indicates that electrode falloff and belt disconnection are currently configured device priority events. In these examples, the HCP interface application is configured to selected or deselect individual priority events based on input received from the HCP specifying selection or deselection of the individual priority events.

Continuing with the example of FIG. 11, the HCP interface application is configured to save and deploy the priority events currently selected in the controls 1104 and 1106. For instance, in some examples, the HCP interface application generates and transmits a new settings request (e.g., the new settings request 572 of FIG. 5D), which triggers the remainder of the process 550 illustrated in FIGS. 5D-5F. It should be noted that, in some examples, the medical device does not require an authorization code to alter the currently configured priority events for an ambulatory cardiac device.

Continuing with the example of FIG. 11, the HCP interface application is configured to close the screen 1100 in response to receiving input from the HCP selecting the control 1100.

Turning now to FIG. 9, a computing device 900 is illustrated schematically. As shown in FIG. 9, the computing device includes at least one processor 902, volatile memory 904, one or more interfaces 906, non-volatile memory 908, and an interconnection mechanism 914. The non-volatile memory 908 includes code 910 and at least one data store 912.

In some examples, the non-volatile (non-transitory) memory 908 includes one or more read-only memory (ROM) chips; one or more hard disk drives or other magnetic or optical storage media; one or more solid state drives (SSDs), such as a flash drive or other solid-state storage media; and/or one or more hybrid magnetic and SSDs. In certain examples, the code 910 stored in the non-volatile memory can include an operating system and one or more applications or programs that are configured to execute under the operating system. Alternatively or additionally, the code 910 can include specialized firmware and embedded software that is executable without dependence upon a commercially available operating system. Regardless, execution of the code 910 can result in manipulated data that may be stored in the data store 912 as one or more data structures. The data structures may have fields that are associated through colocation in the data structure. Such associations may likewise be achieved by allocating storage for the fields in locations within memory that convey an association between the fields. However, other mechanisms may be used to establish associations between information in fields of a data structure, including through the use of pointers, tags, or other mechanisms.

Continuing the example of FIG. 9, the processor 902 can be one or more programmable processors to execute one or more executable instructions, such as a computer program specified by the code 910, to control the operations of the computing device 900. As used herein, the term “processor” describes circuitry that executes a function, an operation, or a sequence of operations. The function, operation, or sequence of operations can be hard coded into the circuitry or soft coded by way of instructions held in a memory device (e.g., the volatile memory 904) and executed by the circuitry. In some examples, the processor 902 is a digital processor, but the processor 902 can be analog, digital, or mixed. As such, the processor 902 can execute the function, operation, or sequence of operations using digital values and/or using analog signals. In some examples, the processor 902 can be embodied in one or more application specific integrated circuits (ASICs), microprocessors, digital signal processors (DSPs), graphics processing units (GPUs), neural processing units (NPUs), microcontrollers, field programmable gate arrays (FPGAs), programmable logic arrays (PLAs), or multicore processors. Examples of the processor 902 that are multicore can provide functionality for parallel, simultaneous execution of instructions or for parallel, simultaneous execution of one instruction on more than one piece of data.

Continuing with the example of FIG. 9, prior to execution of the code 910 the processor 902 can copy the code 910 from the non-volatile memory 908 to the volatile memory 904. In some examples, the volatile memory 904 includes one or more static or dynamic random access memory (RAM) chips and/or cache memory (e.g. memory disposed on a silicon die of the processor 902). Volatile memory 904 can offer a faster response time than a main memory, such as the non-volatile memory 908.

Through execution of the code 910, the processor 902 can control operation of the interfaces 906. The interfaces 906 can include network interfaces. These network interfaces can include one or more physical interfaces (e.g., a radio, an ethernet port, a USB port, etc.) and a software stack including drivers and/or other code 910 that is configured to communicate with the one or more physical interfaces to support one or more LAN, PAN, and/or WAN standard communication protocols. The communication protocols can include, for example, TCP and UDP among others. As such, the network interfaces enable the computing device 900 to access and communicate with other computing devices via a computer network.

The interfaces 906 can include user interfaces. For instance, in some examples, the user interfaces include user input and/or output devices (e.g., a keyboard, a mouse, a touchscreen, a display, a speaker, a camera, an accelerometer, a biometric scanner, an environmental sensor, etc.) and a software stack including drivers and/or other code 910 that is configured to communicate with the user input and/or output devices. As such, the user interfaces enable the computing device 900 to interact with users to receive input and/or render output. This rendered output can include, for instance, one or more GUIs including one or more controls configured to display output and/or receive input. The input can specify values to be stored in the data store 912. The output can indicate values stored in the data store 912.

Continuing with the example of FIG. 9, the various features of the computing device 900 described above can communicate with one another via the interconnection mechanism 914. In some examples, the interconnection mechanism 914 includes a communications bus.

The teachings of the present disclosure can be generally applied to external medical monitoring and/or treatment devices that include one or more sensors as described herein. Such external medical devices can include, for example, ambulatory medical devices as described herein that are capable of and designed for moving with the patient as the patient goes about his or her daily routine. An example ambulatory medical device can be a wearable medical device such as a WCD, a wearable cardiac monitoring device, an in-hospital device such as an HWD, a short-term wearable cardiac monitoring and/or therapeutic device, mobile cardiac event monitoring devices, and other similar wearable medical devices.

The wearable medical device can be capable of continuous use by the patient. In some implementations, the continuous use can be substantially or nearly continuous in nature. That is, the wearable medical device can be continuously used, except for sporadic periods during which the use temporarily ceases (e.g., while the patient bathes, while the patient is refit with a new and/or a different garment, while the battery is charged/changed, while the garment is laundered, etc.). Such substantially or nearly continuous use as described herein may nonetheless be considered continuous use. For example, the wearable medical device can be configured to be worn by a patient for as many as 24 hours a day. In some implementations, the patient can remove the wearable medical device for a short portion of the day (e.g., for half an hour to bathe). In such an example, nearly continuous can include 23.5 hours a day of wear with a half hour removal period.

Further, the wearable medical device can be configured as a long term or extended use medical device. Such devices can be configured to be used by the patient for an extended period of several days, weeks, months, or even years. In some examples, the wearable medical device can be used by a patient for an extended period of at least one week. In some examples, the wearable medical device can be used by a patient for an extended period of at least 30 days. In some examples, the wearable medical device can be used by a patient for an extended period of at least one month. In some examples, the wearable medical device can be used by a patient for an extended period of at least two months. In some examples, the wearable medical device can be used by a patient for an extended period of at least three months. In some examples, the wearable medical device can be used by a patient for an extended period of at least six months. In some examples, the wearable medical device can be used by a patient for an extended period of at least one year. In some implementations, the extended use can be uninterrupted until a physician or other healthcare provider (HCP) provides specific instruction to the patient to stop use of the wearable medical device.

Regardless of the extended period of wear, the use of the wearable medical device can include continuous wear by the patient as described above. For example, the continuous use can include continuous wear or attachment of the wearable medical device to the patient, e.g., through one or more of the electrodes as described herein, during both periods of monitoring and periods when the device may not be monitoring the patient but is otherwise still worn by or otherwise attached to the patient. The wearable medical device can be configured to continuously monitor the patient for cardiac-related information (e.g., ECG information, including arrhythmia information, cardio-vibrations, etc.) and/or non-cardiac information (e.g., blood oxygen, the patient's temperature, glucose levels, tissue fluid levels, and/or lung vibrations). The wearable medical device can carry out its monitoring in periodic or aperiodic time intervals or times. For example, the monitoring during intervals or times can be triggered by a user action or another event.

As noted above, the wearable medical device can be configured to monitor other non-ECG physiologic parameters of the patient in addition to cardiac related parameters. For example, the wearable medical device can be configured to monitor, for example, pulmonary-vibrations (e.g., using microphones and/or accelerometers), breath vibrations, sleep related parameters (e.g., snoring, sleep apnea), tissue fluids (e.g., using radio-frequency transmitters and sensors), among others.

Other example wearable medical devices include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the ambulatory medical devices described herein can be pacing-enabled, e.g., capable of providing therapeutic pacing pulses to the patient. In some examples, the ambulatory medical devices can be configured to monitor for and/or measure ECG metrics including, for example, heart rate (such as average, median, mode, or other statistical measure of the heart rate, and/or maximum, minimum, resting, pre-exercise, and post-exercise heart rate values and/or ranges), heart rate variability metrics, premature ventricular contraction (PVC) burden or counts, atrial fibrillation burden metrics, pauses, heart rate turbulence, QRS height, QRS width, changes in a size or shape of morphology of the ECG information, cosine R-T, artificial pacing, QT interval, QT variability, T wave width, T wave alternans, T-wave variability, and ST segment changes.

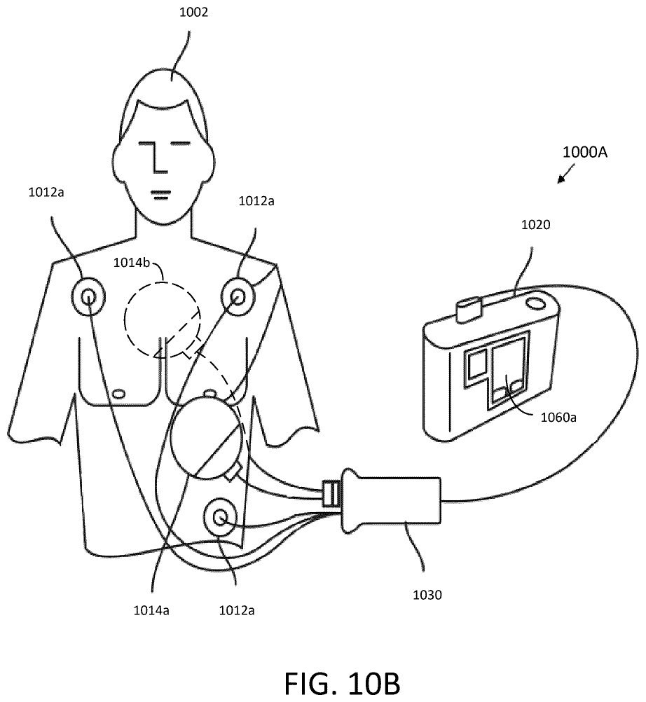

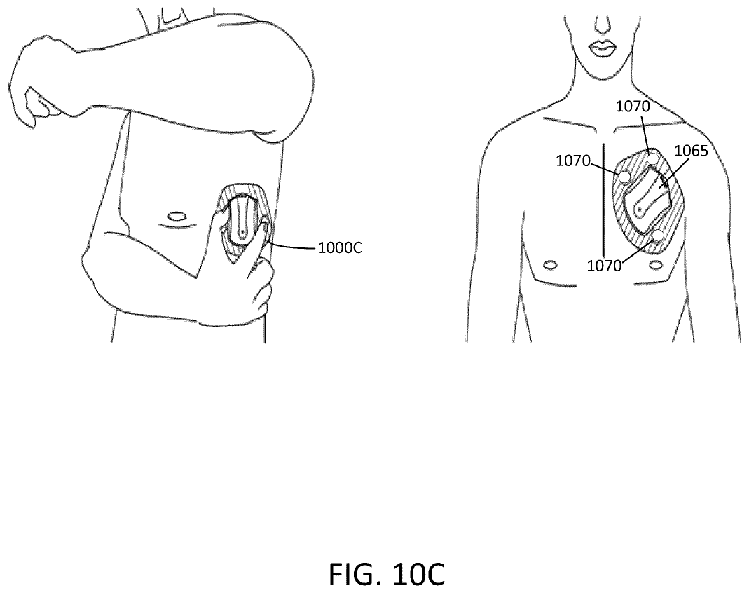

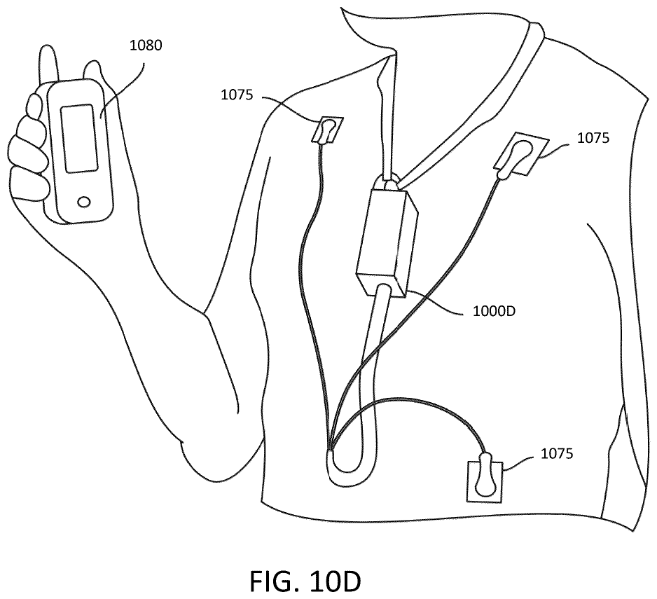

FIG. 10A illustrates an example medical device 1000 that is external, ambulatory, and wearable by a patient 1002, and configured to implement one or more configurations described herein. For example, the medical device 1000 can be a non-invasive medical device configured to be located substantially external to the patient. Such a medical device 1000 can be, for example, an ambulatory medical device that is capable of and designed for moving with the patient as the patient goes about his or her daily routine. For example, the medical device 1000 as described herein can be bodily-attached to the patient such as the LifeVest® wearable cardioverter defibrillator available from ZOLL® Medical Corporation. Such wearable defibrillators typically are worn nearly continuously for two to three months at a time. During the period of time in which they are worn by the patient, the wearable defibrillator can be configured to continuously monitor the vital signs of the patient and, upon determination that treatment is required, can be configured to deliver one or more therapeutic electrical pulses to the patient. For example, such therapeutic shocks can be pacing, defibrillation, or transcutaneous electrical nerve stimulation (TENS) pulses.

The medical device 1000 can include one or more of the following: a garment 1010, one or more ECG sensing electrodes 1012, one or more non-ECG physiological sensors 1013, one or more therapy electrodes 1014a and 1014b (collectively referred to herein as therapy electrodes 1014), a medical device controller 1020 (e.g., controller 400 as described above in the discussion of FIG. 4), a connection pod 1030, a patient interface pod 1040, a belt 1050, or any combination of these. In some examples, at least some of the components of the medical device 1000 can be configured to be affixed to the garment 1010 (or in some examples, permanently integrated into the garment 1010), which can be worn about the patient's torso.

The medical device controller 1020 can be operatively coupled to the sensing electrodes 1012, which can be affixed to the garment 1010, e.g., assembled into the garment 1010 or removably attached to the garment, e.g., using book and loop fasteners. In some implementations, the sensing electrodes 1012 can be permanently integrated into the garment 1010. The medical device controller 1020 can be operatively coupled to the therapy electrodes 1014. For example, the therapy electrodes 1014 can also be assembled into the garment 1010, or, in some implementations, the therapy electrodes 1014 can be permanently integrated into the garment 1010. In an example, the medical device controller 1020 includes a patient user interface 1060 to allow a patient interface with the externally-worn device. For example, the patient can use the patient user interface 1060 to respond to activity related questions, prompts, and surveys as described herein.

Component configurations other than those shown in FIG. 10A are possible. For example, the sensing electrodes 1012 can be configured to be attached at various positions about the body of the patient 1002. The sensing electrodes 1012 can be operatively coupled to the medical device controller 1020 through the connection pod 1030. In some implementations, the sensing electrodes 1012 can be adhesively attached to the patient 1002. In some implementations, the sensing electrodes 1012 and at least one of the therapy electrodes 1014 can be included on a single integrated patch and adhesively applied to the patient's body.

The sensing electrodes 1012 can be configured to detect one or more cardiac signals. Examples of such signals include ECG signals and/or other sensed cardiac physiological signals from the patient. In certain examples, as described herein, the non-ECG physiological sensors 1013 are components such as accelerometers, vibrational sensors, RF-based sensors, and other measuring devices for recording additional non-ECG physiological parameters. For example, as described above, the such non-ECG physiological sensors are configured to detect other types of patient physiological parameters and acoustic signals, such as tissue fluid levels, cardio-vibrations, lung vibrations, respiration vibrations, patient movement, etc.

In some examples, the therapy electrodes 1014 can also be configured to include sensors configured to detect ECG signals as well as other physiological signals of the patient. The connection pod 1030 can, in some examples, include a signal processor configured to amplify, filter, and digitize these cardiac signals prior to transmitting the cardiac signals to the medical device controller 1020. One or more of the therapy electrodes 1014 can be configured to deliver one or more therapeutic defibrillating shocks to the body of the patient 1002 when the medical device 1000 determines that such treatment is warranted based on the signals detected by the sensing electrodes 1012 and processed by the medical device controller 1020. Example therapy electrodes 1014 can include metal electrodes such as stainless-steel electrodes that include one or more conductive gel deployment devices configured to deliver conductive gel to the metal electrode prior to delivery of a therapeutic shock.

In some implementations, medical devices as described herein can be configured to switch between a therapeutic medical device and a monitoring medical device that is configured to only monitor a patient (e.g., not provide or perform any therapeutic functions). For example, therapeutic components such as the therapy electrodes 1014 and associated circuitry can be optionally decoupled from (or coupled to) or switched out of (or switched in to) the medical device. For example, a medical device can have optional therapeutic elements (e.g., defibrillation and/or pacing electrodes, components, and associated circuitry) that are configured to operate in a therapeutic mode. The optional therapeutic elements can be physically decoupled from the medical device to convert the therapeutic medical device into a monitoring medical device for a specific use (e.g., for operating in a monitoring-only mode) or a patient. Alternatively, the optional therapeutic elements can be deactivated (e.g., via a physical or a software switch), essentially rendering the therapeutic medical device as a monitoring medical device for a specific physiologic purpose or a particular patient. As an example of a software switch, an authorized person can access a protected user interface of the medical device and select a preconfigured option or perform some other user action via the user interface to deactivate the therapeutic elements of the medical device.

FIG. 10B illustrates a hospital wearable defibrillator 1000A that is external, ambulatory, and wearable by a patient 1002. Hospital wearable defibrillator 1000A can be configured in some implementations to provide pacing therapy, e.g., to treat bradycardia, tachycardia, and asystole conditions. The hospital wearable defibrillator 1000A can include one or more ECG sensing electrodes 1012a, one or more therapy electrodes 1014a and 1014b, a medical device controller 1020 and a connection pod 1030. For example, each of these components can be structured and function as like number components of the medical device 1000. For example, the electrodes 1012a, 1014a, 1014b can include disposable adhesive electrodes. For example, the electrodes can include sensing and therapy components disposed on separate sensing and therapy electrode adhesive patches. In some implementations, both sensing and therapy components can be integrated and disposed on a same electrode adhesive patch that is then attached to the patient. For example, the front adhesively attachable therapy electrode 1014a attaches to the front of the patient's torso to deliver pacing or defibrillating therapy. Similarly, the back adhesively attachable therapy electrode 1014b attaches to the back of the patient's torso. In an example scenario, at least three ECG adhesively attachable sensing electrodes 1012a can be attached to at least above the patient's chest near the right arm, above the patient's chest near the left arm, and towards the bottom of the patient's chest in a manner prescribed by a trained professional.

A patient being monitored by a hospital wearable defibrillator and/or pacing device may be confined to a hospital bed or room for a significant amount of time (e.g., 75% or more of the patient's stay in the hospital). As a result, a user interface 1060a can be configured to interact with a user other than the patient, e.g., a nurse, for device-related functions such as initial device baselining, setting and adjusting patient parameters, and changing the device batteries. Such interactions can also be accomplished using an HCP interface application (e.g., the HCP interface application 122A of FIG. 1).

In some examples, the hospital wearable defibrillator 1000A can further includes one or more motion sensors such as accelerometers. For example, an accelerometer can be integrated into one or more of a sensing electrode 1012a (e.g., integrated into the same patch as the sensing electrode), a therapy electrode 1014a (e.g., integrated into the same patch as the therapy electrode), the medical device controller 1020, the connection pod 1030, and various other components of the hospital wearable defibrillator 1000A.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, e.g., analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and have a similar configuration as the hospital wearable defibrillator described above in connection with FIG. 10A.

FIGS. 10C and 10D illustrate example wearable patient monitoring devices with no treatment or therapy functions. For example, such devices are configured to monitor one or more physiological parameters of a patient, e.g., for remotely monitoring and/or diagnosing a condition of the patient. For example, such physiological parameters can include a patient's ECG information, tissue (e.g., lung) fluid levels, cardio-vibrations (e.g., using accelerometers or microphones), and other related cardiac information. A cardiac monitoring device is a portable device that the patient can carry around as he or she goes about their daily routine.

Referring to FIG. 10C, an example wearable patient monitoring device 1000C can include tissue fluid monitors 1065 that use RF based techniques to assess fluid levels and accumulation in a patient's body tissue. Such tissue fluid monitors 1065 can be configured to measure fluid content in the lungs, typically for diagnosis and follow-up of pulmonary edema or lung congestion in heart failure patients. The tissue fluid monitors 1065 can include one or more antennas configured to direct RF waves through a patient's tissue and measure output RF signals in response to the waves that have passed through the tissue. In certain implementations, the output RF signals include parameters indicative of a fluid level in the patient's tissue. In examples, device 1000C may be a cardiac monitoring device that also includes digital sensing electrodes 1070 for sensing ECG activity of the patient. Device 1000C can pre-process the ECG signals via one or more ECG processing and/or conditioning circuits such as an ADC, operational amplifiers, digital filters, and/or signal amplifiers under control of a microprocessor. Device 1000C can transmit information descriptive of the ECG activity and/or tissue fluid levels via a network interface to a remote server for analysis. Additionally, in certain implementations, the device 1000C can include one or accelerometers for measuring motion signals as described herein.

Referring to FIG. 10D, another example wearable cardiac monitoring device 1000D can be attached to a patient via at least three adhesive digital cardiac sensing electrodes 1075 disposed about the patient's torso. Additionally, in certain implementations, the device 1000D can include one or accelerometers integrated into, for example, one or more of the digital sensing electrodes for measuring motion signals as described herein.

Cardiac devices 1000C and 1000D are used in cardiac monitoring and telemetry and/or continuous cardiac event monitoring applications, e.g., in patient populations reporting irregular cardiac symptoms and/or conditions. These devices can transmit information descriptive of the ECG activity and/or tissue fluid levels via a network interface to a remote server for analysis. Example cardiac conditions that can be monitored include atrial fibrillation (AF), bradycardia, tachycardia, atrio-ventricular block, Lown-Ganong-Levine syndrome, atrial flutter, sino-atrial node dysfunction, cerebral ischemia, pause(s), and/or heart palpitations. For example, such patients may be prescribed a cardiac monitoring device for an extended period of time, e.g., 10 to 30 days, or more. In some ambulatory cardiac monitoring and/or telemetry applications, a portable cardiac monitoring device can be configured to continuously monitor the patient for a cardiac anomaly, and when such an anomaly is detected, the monitor can automatically send data relating to the anomaly to a remote server. The remote server may be located within a 24-hour manned monitoring center, where the data is interpreted by qualified, cardiac-trained reviewers and/or HCPs, and feedback provided to the patient and/or a designated HCP via detailed periodic or event-triggered reports. In certain cardiac event monitoring applications, the cardiac monitoring device is configured to allow the patient to manually press a button on the cardiac monitoring device to report a symptom. For example, a patient can report symptoms such as a skipped beat, shortness of breath, light headedness, racing heart rate, fatigue, fainting, chest discomfort, weakness, dizziness, and/or giddiness. The cardiac monitoring device can record predetermined physiologic parameters of the patient (e.g., ECG information) for a predetermined amount of time (e.g., 1-30 minutes before and 1-30 minutes after a reported symptom). As noted above, the cardiac monitoring device can be configured to monitor physiologic parameters of the patient other than cardiac related parameters. For example, the cardiac monitoring device can be configured to monitor, for example, cardio-vibrational signals (e.g., using accelerometers or microphones), pulmonary-vibrational signals, breath vibrations, sleep related parameters (e.g., snoring, sleep apnea), tissue fluids, among others.

In some examples, the devices described herein (e.g., FIGS. 10A-10D) can communicate with a remote server via an intermediary or gateway device 1080 such as that shown in FIG. 10D. For instance, devices such as shown in FIGS. 10A-D can be configured to include a network interface communications capability as described herein in reference to, for example, FIG. 4.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the scope of the appended claims. For example, it is to be understood that the present disclosure contemplates that, to the extent possible, one or more features of any embodiment can be combined with one or more features of any other embodiment.

Other examples are within the scope of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

1. A cardiac system for efficiently publishing ECG data for subscription-based access, comprising:
an externally worn cardiac device configured to sense one or more ECG signals from a skin of a patient, the externally worn cardiac device comprising
a memory configured to store a device identifier that uniquely identifies the externally worn cardiac device among a plurality of externally worn cardiac devices, and
at least one processor coupled to the memory, the at least one processor configured to
subscribe to a device-specific topic that relates to one or more device parameters for controlling operation of the externally worn cardiac device, and
publish to a first topic that relates to information derived from the one or more ECG signals sensed by the externally worn cardiac device,
the first topic being distinct from the device-specific topic,
wherein the device-specific topic incorporates the device identifier.

2. The cardiac system of claim 1, wherein the at least one processor is further configured to:
receive, via the device-specific topic, a message specifying one or more device settings associated with the externally worn cardiac device; and
apply the one or more device settings to the one or more device parameters of the externally worn cardiac device.

3. The cardiac system of claim 2, wherein the one or more device settings comprise a localization setting.

4. The cardiac system of claim 3, further comprising a device control service configured to:
receive input specifying the one or more device settings;
generate the message specifying the one or more device settings based on the input; and
publish the message to the device-specific topic.

5. The cardiac system of claim 1, wherein the at least one processor is further configured to:
subscribe to a patient-specific topic that relates to device parameters for controlling operation of the externally worn cardiac device, the patient-specific topic being distinct from the device-specific topic and the first topic;
receive, via the patient-specific topic, a message specifying one or more patient settings associated with the patient; and
apply the one or more patient settings to the one or more device parameters for controlling operation of the externally worn cardiac device.

6. The cardiac system of claim 5, wherein:
the externally worn cardiac device comprises
one or more monitoring electrodes configured to sense the one or more ECG signals, and
one or more therapy electrodes configured to discharge electrotherapy to the patient's skin; and
the one or more patient settings comprise one or more shock settings assigned to the electrotherapy.

7. The cardiac system of claim 6, further comprising a device control service configured to:
receive input specifying the one or more patient settings;
generate the message specifying the one or more patient settings based on the input; and
publish the message to the patient-specific topic.

8. The cardiac system of claim 7, wherein the at least one processor is further configured to:
generate an authentication code; and
verify that the message specifying the one or more patient settings comprises the authentication code.

9. The cardiac system of claim 1, wherein:
the externally worn cardiac device is further configured to sense one or more cardio-acoustic signals from the patient; and
the first topic further relates to information derived from the one or more cardio-acoustic signals sensed by the externally worn cardiac device.

10. The cardiac system of claim 1, wherein:
the externally worn cardiac device is further configured to collect device event data indicating a capability of the externally worn cardiac device to monitor and treat the patient; and
the first topic further relates to information derived from the device event data collected by the externally worn cardiac device.

11. The cardiac system of claim 10, wherein the device event data comprises one or more of a held response button condition, a disconnected therapy electrode condition, or unable to treat condition.

12. The cardiac system of claim 1, wherein:
the at least one processor is further configured to
derive ECG data from the one or more ECG signals,
identify a cardiac arrhythmia condition of the patient indicated within the ECG data, and
transmit, using a first communication protocol, the ECG data to a remote storage service; and
to publish to the first topic comprises to publish, using a second communication protocol distinct from the first communication protocol, a message specifying the cardiac arrhythmia condition of the patient.

13. The cardiac system of claim 12, wherein:
the device-specific topic is a first device-specific topic; and
to transmit the ECG data to the remote storage service comprises to:
publish a message specifying a request for an upload link to a second topic distinct from the first topic and the first device-specific topic,
receive, via a subscription to a second device-specific topic, a message specifying the upload link, the second device-specific topic being distinct from the first topic, the second topic, and the first device-specific topic, and
transmit the ECG data to the remote storage service via the upload link.

14. The cardiac system of claim 12, further comprising the remote storage service, wherein the remote storage service is configured to:
receive the ECG data using the first communication protocol; and
publish a message identifying the ECG data to a second topic using the first communication protocol, the second topic being distinct from the first topic and the device-specific topic.

15. The cardiac system of claim 14, wherein the message specifying the cardiac arrhythmia condition further specifies an identifier of the ECG data.

16. The cardiac system of claim 15, further comprising a message handling service configured to:
receive the message specifying the cardiac arrhythmia condition of the patient;
receive the message identifying the ECG data; and
communicate a notification message to a reporting service in response to reception of the message specifying the cardiac arrhythmia condition of the patient.

17. The cardiac system of claim 16, further comprising the reporting service, wherein the reporting service is configured to:
receive the notification message; and
communicate an alert message to a recipient process, the alert message specifying a link between the cardiac arrhythmia condition of the patient and the ECG data.

18. The cardiac system of claim 17, wherein:
the externally worn cardiac device comprises a security credential created during manufacture of the externally worn cardiac device; and
the message handling service is further configured to
register the security credential, and
authenticate communications from the externally worn cardiac device using the security credential.

19. The cardiac system of claim 18, wherein:
the security credential comprises the device identifier that uniquely identifies the externally worn cardiac device among the plurality of externally worn cardiac devices; and
the message handling service is further configured to identify the externally worn cardiac device using the device identifier.

20. The cardiac system of claim 12, wherein the first communication protocol is hypertext transfer protocol (HTTP) and the second communication protocol is message queuing telemetry transport (MQTT).

21.-63. (canceled)
